(19) United States
(12) Patent Application Publication
(10) Pub. No.: US 2025/0090819 A1
(43) Pub. Date: Mar. 20, 2025

(54) DEVICES AND METHODS FOR SHUNT EVALUATION

(71) Applicant: Dignity Health, San Francisco, CA (US)

(72) Inventors: Steve Sungwon Cho, San Francisco, CA (US); Dakota Graham, San Francisco, CA (US)

(21) Appl. No.: 18/726,513

(22) PCT Filed: Jan. 5, 2023
(86) PCT No.: PCT/US23/60182

§ 371 Date: Jul. 3, 2024

Related U.S. Application Data
(60) Provisional application No. 63/296,783, filed on Jan. 5, 2022.

(51) Int. Cl. A61M 27/00

(52) U.S. Cl. CPC A61M 27/006; A61M 2205/0288; A61M 2205/32; A61M 2205/3334; A61M 2205/3375

(57) ABSTRACT

An implantable valve device includes a valve unit having a flow limiter in operative association with a dial for controlling a flow of material through a lumen in communication with the valve unit. The dial includes a dial indicator viewable through cross-sectional imaging for determining and/or otherwise verifying a “pressure setting” of the valve unit. The valve unit can include a housing that encapsulates the dial therein and provides one or more reference indicators that enable a practitioner to determine an orientation of the valve unit and further clarify the position of the dial indicator. The valve unit can be interrogated through conventional non-invasive cross-sectional imaging methods such as ultrasound.

### CROSS-REFERENCE TO RELATED APPLICATIONS

This is a PCT Patent Application that claims benefit to U.S. Provisional Patent Application Ser. No. 63/296,783 filed 5 Jan. 2022, which is herein incorporated by reference in its entirety.

### FIELD

The present disclosure generally relates to surgical shunts, and in particular, to devices and methods for shunt evaluation through non-invasive cross-sectional imaging.

### BACKGROUND

Most modern commercial surgical shunts have adjustable pressure settings that allow physicians and other providers to noninvasively adjust the pressure settings as needed. However, the only reliable and recordable method of confirming the pressure setting on current commercial shunts are through x-ray, a form of ionizing radiation that can harm patients over repeated exposures. Not properly confirming shunt pressure settings can be devastating and life-threatening; for example, in neurosurgical applications, patients can develop hydrocephalus and/or over-shunting which can lead to subdural hematomas. Thus, patients with shunts can be exposed to countless x-rays over their lifetime, which is especially concerning in pediatric patients, where shunt placement takes up an even larger portion of neurosurgical cases.

Furthermore, patients with shunts utilize healthcare frequently due to concerns for shunt failure. The only definitive method of confirming shunt patency is to perform surgery to expose and test the shunt. Other less invasive methods are either inaccurate, expensive, and/or result in significant radiation exposure.

It is with these observations in mind, among others, that various aspects of the present disclosure were conceived and developed.

### BRIEF DESCRIPTION OF THE DRAWINGS

FIG. 1A is an illustration showing a perspective view of a valve device having a valve unit according to aspects of the present disclosure;

FIG. 1B is an illustration showing the valve device of FIG. 1A implanted within the body and being interrogated by an ultrasound device;

FIG. 2A is an illustration showing a cross-sectional perspective view of the valve device of FIG. 1A having a first embodiment of a valve unit;

FIG. 2B is an illustration showing a top plan view of one embodiment of the valve device of FIG. 2A with a reference marker in a first arrangement;

FIG. 2C is an illustration showing a top plan view of one embodiment of the valve device of FIG. 2A with a reference marker in a second arrangement;

FIG. 3A is an illustration showing a top perspective view of a housing of the valve unit of FIG. 2A;

FIG. 3B is an illustration showing a bottom perspective view of the housing of FIG. 3A;

FIG. 3C is an illustration showing a cross-sectional perspective view of the housing of FIG. 3A taken along line 3C-3C of FIG. 3A;

FIG. 3D is an illustration showing a cross-sectional side view of the housing of FIG. 3A taken along line 3D-3D of FIG. 3A;

FIG. 3E is an illustration showing a full cross-sectional perspective view of the housing of FIG. 3A taken along line 3D-3D of FIG. 3A;

FIG. 4A is an illustration showing a top perspective view of a dial of the valve unit of FIG. 2A;

FIG. 4B is an illustration showing a bottom perspective view of the dial of FIG. 4A;

FIG. 4C is an illustration showing a bottom perspective view of the dial of FIG. 4B coupled with a flow control unit;

FIG. 5 is an illustration showing a locking mechanism of the valve unit of FIG. 2A including engagement of the dial with the housing;

FIG. 6A is an illustration showing a top perspective view of the valve unit of FIG. 2A with the housing in phantom;

FIG. 6B is an illustration showing a cutaway side view of the valve unit of FIG. 6A being adjusted by an external magnetic device;

FIG. 6C is an illustration showing a cross-sectional side view of the valve unit of FIG. 6A;

FIG. 7A is an illustration showing a cross-sectional side view of the valve unit of FIG. 6C with boxes indicating areas visible under cross-sectional imaging;

FIG. 7B is an illustration showing a cross-sectional image of the valve unit of FIG. 7A captured by an ultrasound device with boxes indicating corresponding areas with respect to FIG. 7A;

FIG. 8A is an illustration showing an example embodiment of a dial indicator of the dial of FIG. 2A;

FIGS. 8B-8K are a series of illustrations showing example shapes of dial indicator surfaces of the dial of FIG. 2A;

FIG. 9 is an illustration showing a cross-sectional perspective view of the valve device of FIG. 1A having a second embodiment of a valve unit;

FIG. 10A is an illustration showing a top perspective view of a housing of the valve unit of FIG. 9;

FIG. 10B is an illustration showing a cross-sectional perspective view of the housing taken along line 10B-10B of FIG. 10A;

FIG. 10C is an illustration showing a cross-sectional side view of the housing taken along line 10C-10C of FIG. 10A;

FIG. 10D is an illustration showing a cross-sectional perspective view of the housing taken along line 10D-10D of FIG. 10A;

FIG. 11A is an illustration showing a top perspective view of a dial of the valve unit of FIG. 9;

FIG. 11B is an illustration showing a bottom perspective view of the dial of FIG. 11A;

FIG. 11C is an illustration showing a bottom perspective view of the dial of FIG. 11B coupled with a flow control unit;

FIG. 12 is an illustration showing a locking mechanism of the valve unit of FIG. 9 including engagement of the dial with the housing;

FIG. 13A is an illustration showing a top perspective view of the valve unit of FIG. 9 with the housing in phantom;

FIG. 13B is an illustration showing a first cutaway side view of the valve unit of FIG. 13A being adjusted by an external magnetic device;

FIG. 13C is an illustration showing a second cutaway side view of the valve unit of FIG. 13A with a top surface of the housing removed;

FIG. 13D is an illustration showing an exploded view of the valve unit of FIG. 13A;

FIG. 13E is an illustration showing a cross-sectional side view of the valve unit of FIG. 13A;

FIG. 14A is an illustration showing a cross-sectional side view of the valve unit of FIG. 13A with the dial in a first position and a dial indicator indicating the first position of the dial;

FIG. 14B is an illustration showing a cross-sectional side view of the valve unit of FIG. 14A with the dial in a second position and the dial indicator indicating the second position of the dial;

FIG. 14C is an illustration showing a cross-sectional side view of the valve unit of FIG. 14A with the dial in a fourth position and the dial indicator indicating the fourth position of the dial;

FIG. 14D is an illustration showing a cross-sectional side view of the valve unit of FIG. 14A with the dial in a sixth position and the dial indicator indicating the sixth position of the dial;

FIG. 15A is an illustration showing an alternate arrangement of the valve unit of FIG. 13A;

FIG. 15B is an illustration showing a cross-sectional side view of the valve unit of FIG. 15A with boxes indicating areas visible under cross-sectional imaging;

FIG. 15C is an illustration showing a cross-sectional image of the valve unit of FIG. 15A captured by an ultrasound device with boxes indicating corresponding areas with respect to FIG. 15B;

FIG. 16A is an illustration showing a cross-sectional perspective view of a first embodiment of a flow indicator within a flow indicator chamber of the valve device of FIG. 1A;

FIG. 16B is an illustration showing a perspective view of the flow indicator of the valve device of FIG. 16A;

FIG. 16C is an illustration showing a cross-sectional side view of the flow indicator within the flow indicator chamber of FIG. 16A when no material is flowing through a distal lumen in communication with the flow indicator chamber;

FIG. 16D is an illustration showing a cross-sectional side view of the flow indicator within the flow indicator chamber of FIG. 16A when material is flowing through a distal lumen in communication with the flow indicator chamber;

FIG. 17A is an illustration showing a cross-sectional side view of a second embodiment of a flow indicator within a flow indicator chamber of the valve device of FIG. 1A in a non-occluding position;

FIG. 17B is an illustration showing an example image that can be captured by an ultrasound device when the flow indicator of FIG. 17A is in the non-occluding position;

FIG. 17C is an illustration showing a cross-sectional side view of the flow indicator within the flow indicator chamber of FIG. 17A when the flow indicator is actuated into a distal lumen in communication with the flow indicator chamber for interrogation;

FIG. 17D is an illustration showing a cross-sectional side view of the flow indicator within the flow indicator chamber of FIG. 17A when the flow indicator is fully inserted into a distal lumen in communication with the flow indicator chamber for interrogation;

FIG. 17E is an illustration showing a cross-sectional side view of the flow indicator within the flow indicator chamber of FIG. 17A when material is not flowing through a distal lumen in communication with the flow indicator chamber;

FIG. 17F is an illustration showing an example image that can be captured of the flow indicator of FIG. 17E by an ultrasound device;

FIG. 17G is an illustration showing a cross-sectional side view of the flow indicator within the flow indicator chamber of FIG. 17A when material not flowing through a distal lumen in communication with the flow indicator chamber;

FIG. 17H is an illustration showing an example image that can be captured of the flow indicator of FIG. 17G by an ultrasound device;

FIG. 17I is an illustration showing returning the flow indicator of FIG. 17G to the non-occluding position of FIG. 17A by an external magnetic device;

FIG. 18A is an illustration showing a testing setup for testing visibility of various dial indicator surfaces and reference indicator surfaces by an ultrasound device;

FIG. 18B is an illustration showing a testing setup for testing visibility of various dial indicator surfaces and reference indicator surfaces by an ultrasound device underneath simulated tissue (chicken breast); and

FIG. 19 is a process flow diagram showing an example method for interrogation and adjustment of the valve device of FIG. 1A.

Corresponding reference characters indicate corresponding elements among the view of the drawings. The headings used in the figures do not limit the scope of the claims.

### DETAILED DESCRIPTION

An implantable valve device includes a valve unit having a flow limiter in operative association with a dial for controlling a flow of material through a lumen in communication with the valve unit. The dial controls the flow limiter and includes a dial indicator viewable through cross-sectional imaging for determining and/or otherwise verifying a “pressure setting” of the valve unit. The dial is moveable between a plurality of positions corresponding with respective “pressure settings” of the flow limiter which can be determined based on a position of the dial indicator as observed through cross-sectional imaging. The valve unit can include a housing that encapsulates the dial therein and provides one or more reference indicators that enable a practitioner to determine an orientation of the valve unit and further clarify the position of the dial indicator (e.g., by comparing the position of the dial indicator to the reference indicator). The valve unit can be interrogated through conventional non-invasive cross-sectional imaging methods (e.g., ultrasound), as opposed to current technologies that require x-ray imaging for interrogation.

In one aspect, a “pressure setting” of the valve unit can be adjusted by rotating or otherwise altering a position of the dial to adjust a pressure applied at the flow limiter. The valve unit can include a locking mechanism that can prevent rotation of the dial by default but can be disengaged to enable adjustment of the position of the dial. In some embodiments, the dial can include a first magnetic-responsive element that, when activated by an external magnetic device, can disengage the locking mechanism and enable a practitioner to adjust a position of the dial. The dial can further include a second magnetic-responsive element that, when activated by an external magnetic device, can enable a practitioner to rotate the dial to a new position of the plurality of positions of the dial. The dial can include a cam that interfaces with the flow limiter such that the position of the dial determines the “pressure setting” of the flow limiter.

In a further aspect, the valve unit can include a flow indicator that is positionable within a lumen distal to the dial. The flow indicator can be moveable by flow of material through the lumen and is similarly viewable through cross-sectional imaging of the flow indicator. In some embodiments, the flow indicator can be configured for temporary positioning within the lumen so as not to occlude flow of material through the lumen during normal operation.

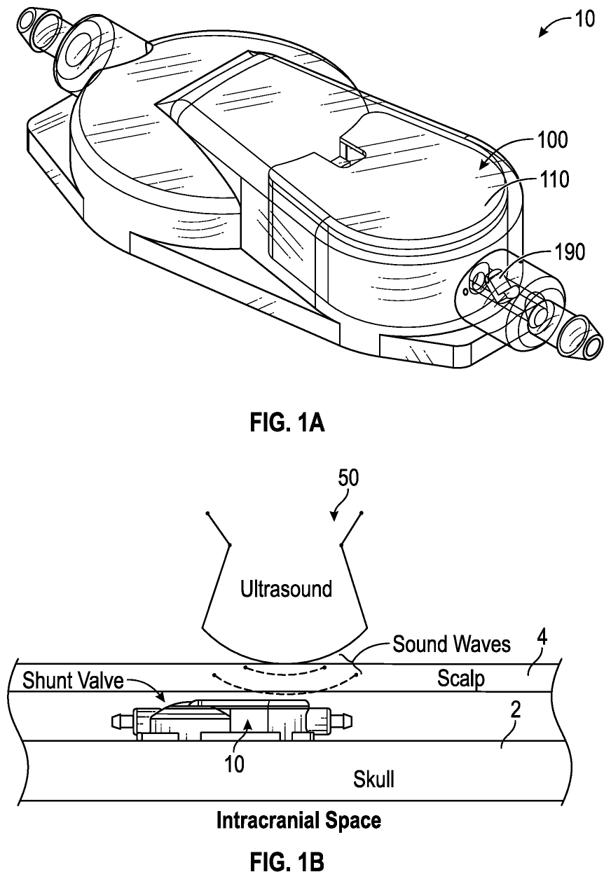

FIG. 1A shows a valve device 10 having a valve unit 100 that can be interrogated by non-invasive cross-sectional imaging techniques. As shown, the valve unit 100 can include a housing 110 and a flow indicator 190 positioned within a distal lumen distal to the housing 110. FIG. 1B shows a side view of one embodiment of the valve device 10 implanted within a body and being interrogated by an ultrasound device 50; the valve device 10 can be positioned between a bodily structure (such as skull 2) and an external surface (such as scalp 4) of the body. When interrogating the valve device 10, probing sound waves emitted by ultrasound device 50 can be directed towards the valve device 10 as shown to obtain a cross-sectional image of the valve device 10.

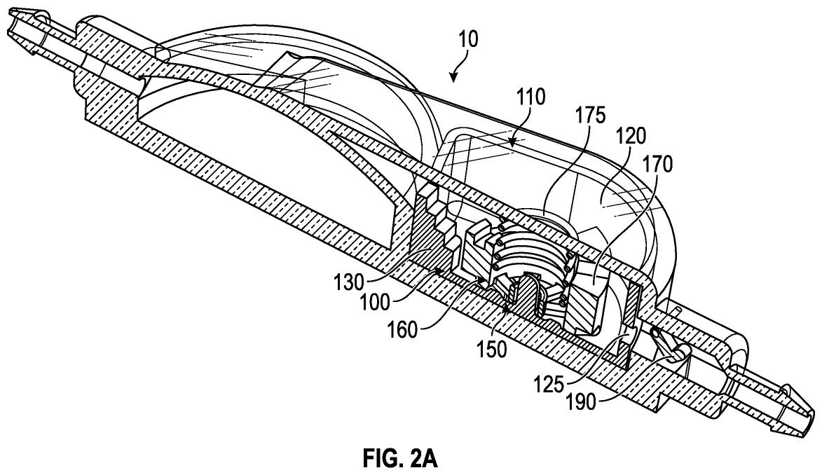

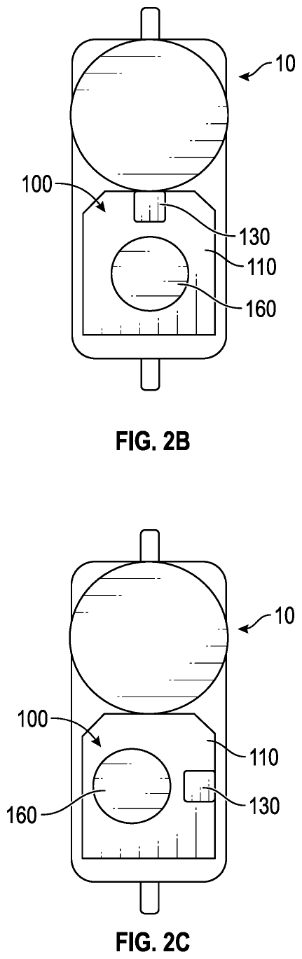

FIG. 2A shows a cross-sectional view of the valve device 10 and valve unit 100 of FIGS. 1A and 1B. As shown, the housing 110 includes a cavity 120 that receives a dial 160 therein for controlling a flow of material through the valve unit 100 and for indicating a “pressure setting” of the valve unit 100 when observed through non-invasive cross-sectional imaging techniques such as ultrasound. The dial 160 is moveable within the housing 110 between a plurality of positions; the position of the dial 160 corresponds with the “pressure setting” of the valve unit 100. The dial 160 includes one or more dial indicators 170 that indicate the position of the dial 160 (and therefore the “pressure setting” of the valve unit 100) and can be observed through cross-sectional imaging. The housing 110 includes one or more reference indicators 130 that can aid a practitioner in determining an orientation of the valve unit 100 and determining a position of the one or more dial indicators 170 to ensure correct interpretation of the “pressure setting” of the valve unit 100. As further shown, and as will be discussed in greater detail below, the valve unit 100 can include a locking mechanism 150 having or otherwise associated with a tensioning element 175 that prevents unintentional alteration of the position of the dial 160. FIG. 2A further shows an outlet 125 of the valve unit 100 and the flow indicator 190 that indicates flow of material though the valve unit 100 when observed through non-invasive cross-sectional imaging techniques such as ultrasound.

FIGS. 2B and 2C show simplified illustrations of the valve unit 100, where FIG. 2C shows one example valve unit 100 having reference indicators 130 in a first arrangement within the housing 110 relative to the dial 160 and FIG. 2C shows another example valve unit 100 having reference indicators 130 in a second arrangement within the housing 110 relative to the dial 160.

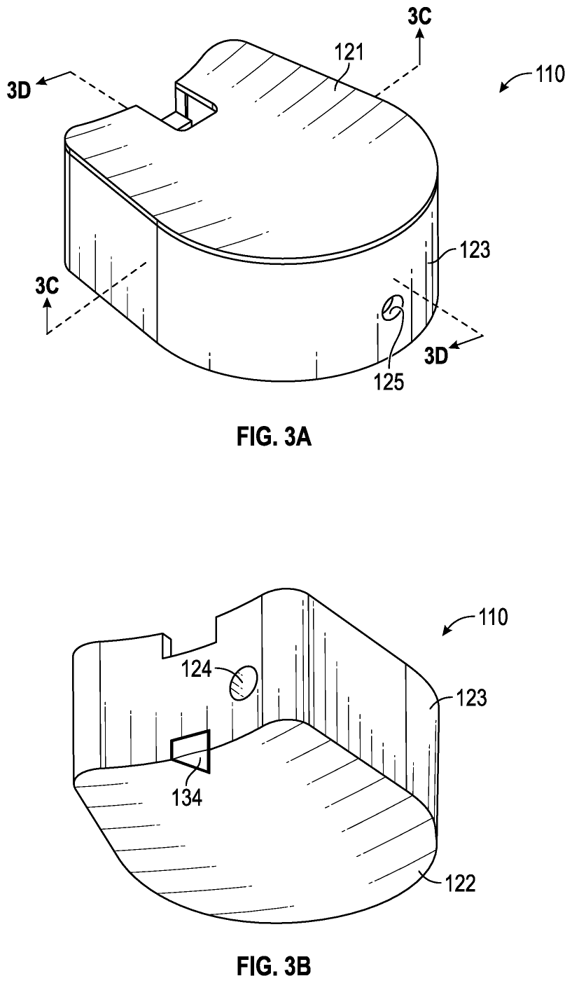

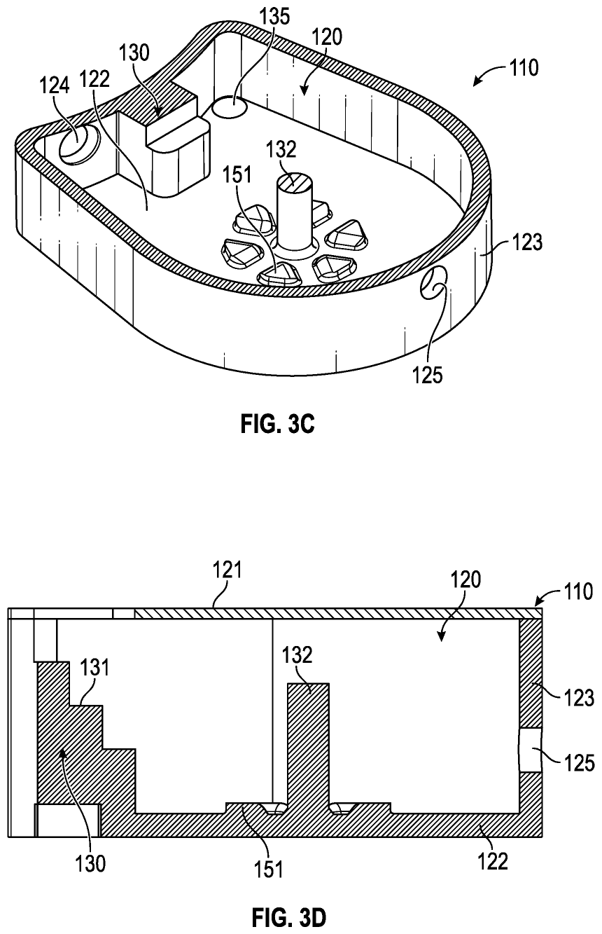

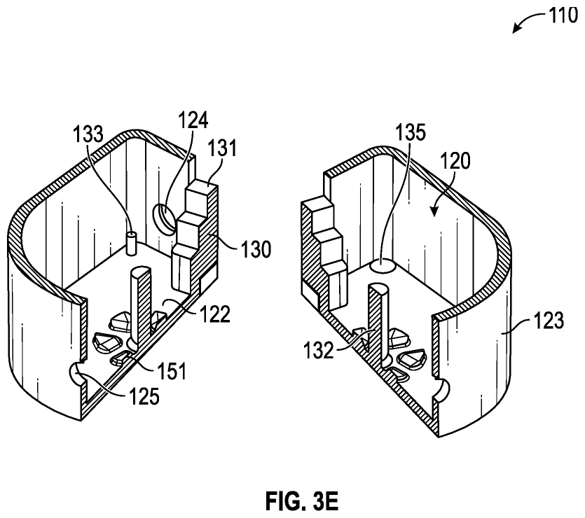

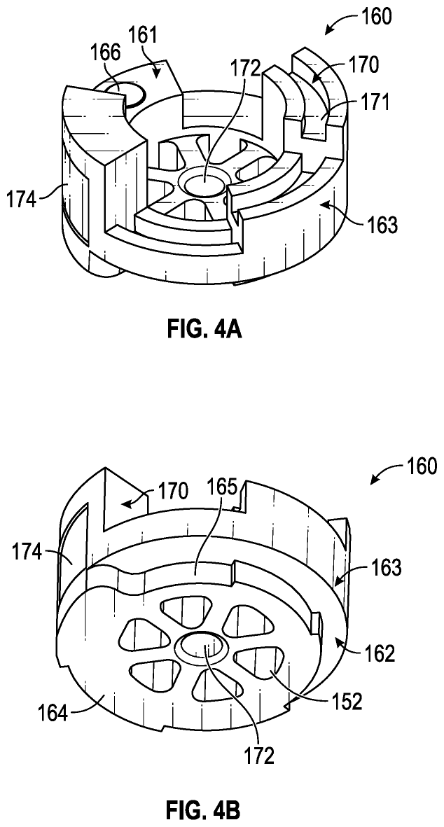

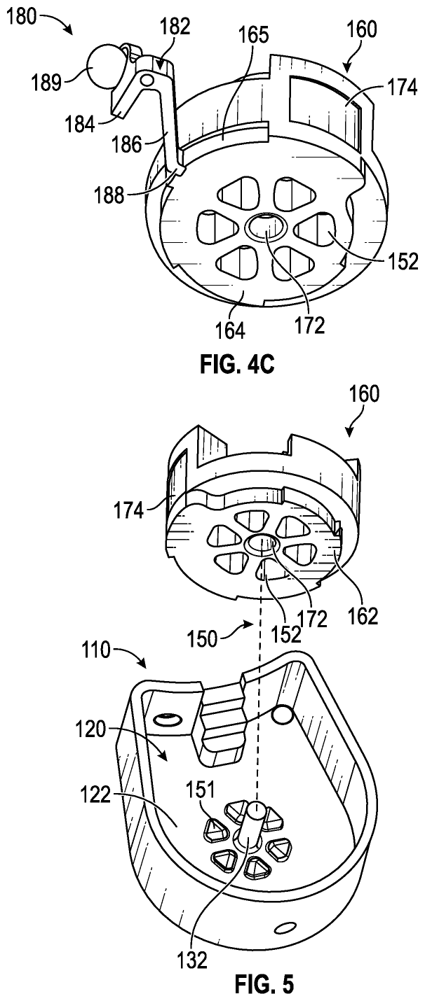

FIGS. 3A-3E illustrate the housing 110 of the valve unit 100 including the cavity 120 that receives the dial 160 (FIG. 2A) therein. As shown, the housing 110 can include a top surface 121, a bottom surface 122 defined opposite from the top surface 121, and sidewalls 123 between the top surface 121 and the bottom surface 122 that define the outlet 125. The housing 110 further defines an inlet 124 formed generally opposite from the outlet 125; in operation, material enters the housing 110 at the inlet 124 and exits the housing 110 at the outlet 125. As shown in FIGS. 3B and 3C, the housing 110 can include one or more radiopaque elements (e.g., first radiopaque element 134 and second radiopaque element 135) that enable aspects of the valve unit 100 to be detectable under x-ray in addition to cross-sectional imaging. FIGS. 3C-3E show the housing 110 having the reference indicators 130; in particular, the reference indicators 130 can include a plurality of reference indicator surfaces 131 that can be configured in a stepped arrangement as shown. When viewed in cross-section from the side of the housing 110, the varying vertical positions of each reference indicator surface 131 are clearly visible and remain stationary relative to the housing 110; this aspect enables practitioners to use the reference indicators 130 as reference to determine a position of the dial 160, and thereby the “pressure setting” enforced by the dial 160. The housing 110 can also include a dial stem 132 that receives the dial 160 in an arrangement that will be discussed in greater detail below. The housing 110 can further include one or more locking mechanism protrusions 151 of the locking mechanism 150 (FIG. 5) that also receive the dial 160 in an arrangement that will be discussed in greater detail below. In the example shown, the locking mechanism protrusions 151 surround the dial stem 132. In a further aspect, with reference to FIG. 3E, the housing 110 can include a flow lever stem 133 positioned near the inlet 124 for receipt of a flow lever 182 (FIG. 4C) in an arrangement that will be discussed in greater detail below.

FIGS. 4A-4C show the dial 160 of the valve unit 100 that can be encapsulated within the housing 110 as discussed above and as shown in the cross-sectional image of FIG. 2A. The dial 160 is moveable between the plurality of positions within the housing 110 and controls the flow of material through the valve unit 100; in particular, the dial 160 can be rotated between the plurality of positions to select a “pressure setting” for the flow of material though the valve unit 100. As shown, the dial 160 includes a top surface 161, a bottom surface 162 defined opposite the top surface 161, and a sidewall 163 between the top surface 161 and the bottom surface 162. The dial 160 further includes a dial aperture 172 that receives the dial stem 132 of the housing 110 when the dial 160 is coupled with the housing. The top surface 161 can include the dial indicators 170 that indicate the position of the dial 160 when viewed through cross-sectional imaging. The dial indicators 170 include a plurality of dial indicator surfaces 171 that each define a unique vertical position and/or a unique cross-sectional shape when viewed through cross-sectional imaging. As the dial 160 rotates within the housing 110 between different positions (e.g., between different angular positions), different dial indicator surfaces 171 become visible through cross-sectional imaging. In the example shown in FIG. 4A, the dial indicators 170 include six unique dial indicator surfaces 171; the positions of these dial indicator surfaces 171 relative to the housing 110 (including the reference indicator surfaces 131 of the housing 110) can inform practitioners as to the position of the dial 160 and thereby the “pressure setting” of the valve unit 100. Note that while six unique dial indicator surfaces 171 are shown in FIGS. 4A-4C, other embodiments are contemplated and shown herein that include more or fewer unique dial indicator surfaces 171 to corresponding “pressure settings”. In a further aspect, the dial 160 can also include a third radiopaque element 166 that enables a practitioner to determine a position of the dial 160 through x-ray imaging.

FIG. 4B shows the bottom surface 162 of the dial 160. As shown, the bottom surface 162 includes one or more locking mechanism recesses 152 of the locking mechanism 150 (FIG. 5) that receive the one or more locking mechanism protrusions 151 (FIGS. 3C-3E) positioned along the bottom surface 122 of the housing 110. In a further aspect, the bottom surface 162 of the dial 160 includes a cam 164 having a plurality of cam surfaces 165 as shown. With additional reference to FIG. 4C, the cam 164 of the dial 160 engages a flow control unit 180 that controls a flow of material that enters the housing 110 through the inlet 124 (FIG. 3E). In the example shown, each cam surface 165 of the plurality of cam surfaces 165 of the dial 160 corresponds with a respective dial indicator surface 171 of the plurality of dial indicator surfaces 171. The flow control unit 180 can include the flow lever 182 in operative association with a cam follower 188 that contacts a cam surface 165 of the plurality of cam surfaces 165 as shown. The flow lever 182 can also include a flow occluder 189 positioned opposite from the cam follower 188 that contacts the inlet 124 of the housing 110 at selective pressure settings to control the flow of material through the inlet 124. In the embodiment shown, the cam surface 165 occupied by the cam follower 188 determines a pressure applied at the inlet 124 by the flow lever 182; as such, the cam surface 165 occupied by the cam follower 188 corresponds directly with the position of the dial 160. In some embodiments, the flow lever 182 can include a first lever arm 184 and a second lever arm 186 that couple at the flow lever stem 133 (FIG. 3E) of the housing 110.

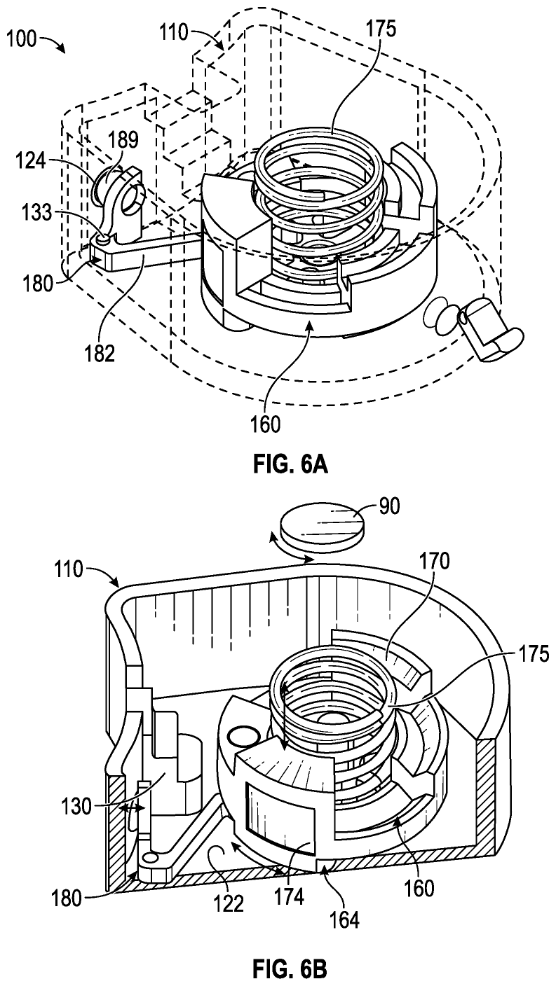

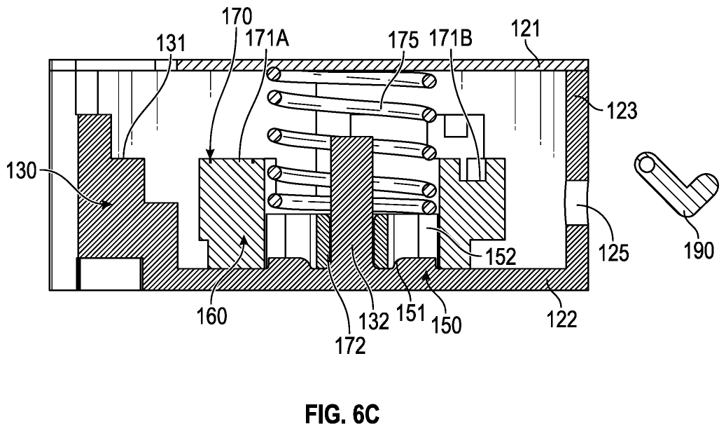

As shown, the dial 160 can include a magnetic-responsive element 174 that, when influenced by external application of a magnetic force by an external magnetic device 90 (FIG. 6B), enables alteration of the position of the dial 160. In particular, when influenced by an external magnetic device 90, the magnetic-responsive element 174 can be attracted to or repulsed by the external magnetic device 90 and can thus be used to rotate or otherwise move the dial 160 to a desired position of the plurality of positions of the dial 160. Rotation of the dial 160 to a new position will also cause the cam follower 188 of the flow control unit 180 to occupy a new cam surface 165 of the cam 164 of the dial 160, which changes the “pressure setting” of the valve unit 100.

FIG. 5 shows engagement of the dial 160 with the housing 110 at a locking mechanism 150 of the valve unit 100. As discussed, the housing 110 can include the locking mechanism protrusions 151 of the locking mechanism 150 along the bottom surface 122 of the housing 110 and surrounding the dial stem 132. The housing 110 is shown in FIG. 5 with the top surface 121 removed. The dial 160 can include the locking mechanism recesses 152 of the locking mechanism 150 along the bottom surface 162 of the dial 160 and surrounding the dial aperture 172. As illustrated, the dial 160 couples with the housing 110 by insertion of the dial stem 132 of the housing 110 into the dial aperture 172 of the dial 160. The locking mechanism recesses 152 along the dial 160 engage the locking mechanism protrusions 151 along the housing 110 as shown. When the magnetic-responsive element 174 is not being activated by the external magnetic device 90 (e.g., absent external application of the magnetic force by the external magnetic device 90), the bottom surface 162 of the dial 160 rests against the bottom surface 122 of the housing 110 and the locking mechanism protrusions 151 couple with the locking mechanism recesses 152 to prevent rotation of the dial 160 within the housing 110. When the magnetic-responsive element 174 is being activated by the external magnetic device 90, the bottom surface 162 of the dial 160 lifts away from the bottom surface 122 of the housing 110 to deactivate the locking mechanism 150 such that the locking mechanism protrusions 151 decouple from the locking mechanism recesses 152 to allow rotation of the dial 160 within the housing 110. With the locking mechanism 150 deactivated, the dial 160 can be rotated to a new position by the external magnetic device 90 interacting with the magnetic-responsive element 174.

FIGS. 6A-6C show the valve unit 100 including the dial 160, the flow control unit 180 and the housing 110. As discussed, the dial aperture 172 of the dial 160 receives the dial stem 132 of the housing 110. The cam follower 188 of the flow control unit 180 contacts the cam 164 of the dial 160 to selectively control a flow of material through the valve unit 100; the “pressure setting” of the valve unit 100 depends on which cam surface 165 of the cam 164 is occupied by the cam follower 188 of the flow control unit 180. The flow lever 182 of the flow control unit 180 couples with the flow lever stem 133 of the housing 110 as shown. In some embodiments, the flow lever 182 can include the flow occluder 189 that contacts the inlet 124 of the housing 110 at variable pressure (e.g., dependent upon which cam surface 165 of the cam 164 is occupied by the cam follower 188) to control the flow of material through the valve unit 100. As further shown, the dial 160 can include the tensioning element 175 positioned between the top surface 161 of the dial 160 and the top surface 121 of the housing 110. The tensioning element 175 ensures that the bottom surface 162 of the dial 160 rests against the bottom surface 122 of the housing 110 and the locking mechanism protrusions 151 couple with the locking mechanism recesses 152 to prevent rotation of the dial 160 within the housing 110 when the magnetic-responsive element 174 is not being activated by the external magnetic device 90. When the magnetic-responsive element 174 is being activated by the external magnetic device 90 as shown in FIG. 6B, the tensioning element 175 can compress to enable the bottom surface 162 of the dial 160 to lift up from the bottom surface 122 of the housing 110 and deactivate the locking mechanism 150 such that the locking mechanism protrusions 151 decouple from the locking mechanism recesses 152. To rotate the dial 160 and alter the setting of the dial 160, a practitioner can lift the dial 160 away from the bottom surface 122 of the housing 110 by the external magnetic device 90 and rotate the external magnetic device 90 in a clockwise or counterclockwise direction such that the magnetic-responsive element 174 follows the external magnetic device 90, thereby rotating the dial 160 in the clockwise or counterclockwise direction. This motion likewise causes the flow occluder 189 of the flow control unit 180 to adjust a pressure being applied at the inlet 124 of the housing 110.

FIG. 6C shows a cross-sectional side view of the valve unit 100. The dial 160 is shown in a position of the plurality of positions of the dial 160 identifiable by positions of a first dial indicator surface 171A and a second dial indicator surface 171B of the dial indicator 170 as shown. In the example, the first dial indicator surface 171A is at a first height and has a first shape (solid block across); the second dial indicator surface 171B is at a second height and has a second shape (notched top). The respective heights and shapes of the first dial indicator surface 171A and the second dial indicator surface 171B are visible under cross-sectional imaging, along with the reference indicators 130 of the housing 110 which can be used to verify an orientation of the valve unit 100 and discern the position of the dial 160 (e.g., by comparison of the heights and shapes of the first dial indicator surface 171A and the second dial indicator surface 171B with the reference indicator surfaces 131 of the reference indicators 130). Further, the flow indicator 190 is shown positioned distal to the outlet 125 of the housing 110.

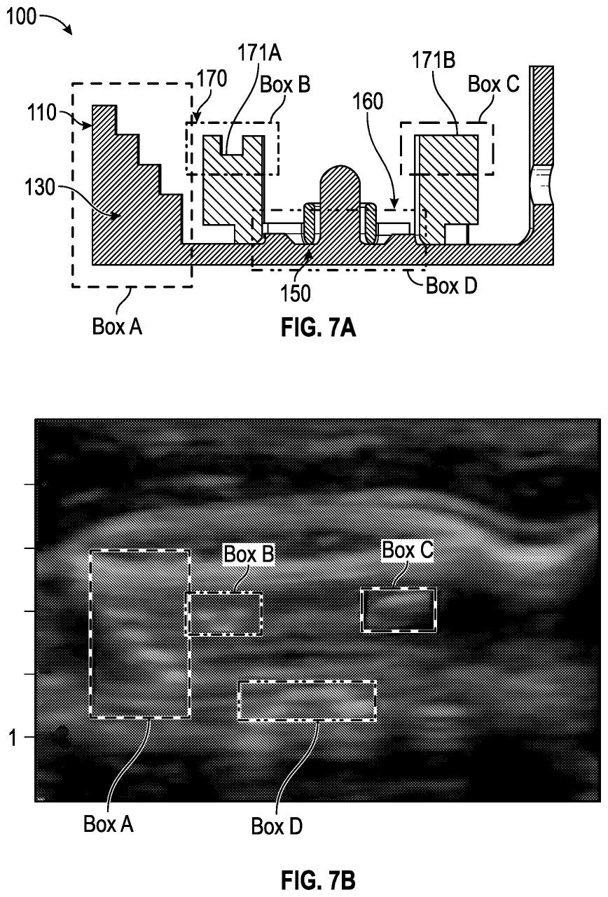

FIGS. 7A and 7B show an example cross-sectional view of the valve unit 100, along with a corresponding cross-sectional image of the housing 110 and the dial 160 captured by ultrasound. The reference indicators 130 are indicated at Box A of FIG. 7A and FIG. 7B in both images. A first dial indicator surface 171A and a second dial indicator surface 171B of the dial indicator 170 are respectively visible at Box B and Box C of FIG. 7A and FIG. 7B. Note that the first dial indicator surface 171A includes a notched top; this is well-distinguishable within the captured cross-sectional image of FIG. 7B from the second dial indicator surface 171B, which shows a solid top in FIG. 7B. The locking mechanism 150 is also visible in both FIGS. 7A and 7B, indicated by Box D in both images. A practitioner can determine the position of the dial 160, and thereby the “pressure setting” of the valve unit 100 by identifying relative positions of the reference indicators 130 and the first dial indicator surface 171A and the second dial indicator surface 171B of the dial indicator 170 within the cross-sectional image captured by ultrasound. The practitioner can determine an orientation of the valve unit 100 by observing which side the reference indicators 130 are on, and can then determine the “pressure setting” of the valve unit 100 by comparing heights and shapes of the first dial indicator surface 171A and the second dial indicator surface 171B of the dial indicator 170 relative to one another and relative to the reference indicators 130.

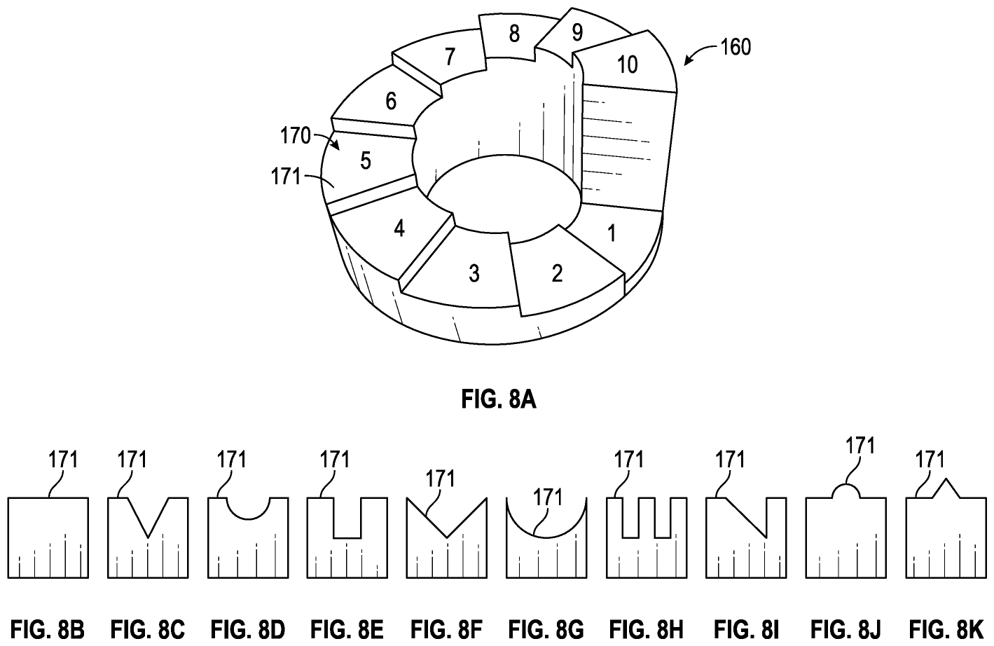

FIGS. 8A-8K show example dial indicators of the dial 160. FIG. 8A shows one example dial 160 having ten settings and ten dial indicator surfaces 171; when viewed in cross-section, a first dial indicator surface and a corresponding second dial indicator surface would be at opposite “sides” of the dial 160 (e.g., if the first dial indicator surface on the “left” is surface #5 when captured under cross-sectional image, then the second dial indicator surface on the “right” would be surface #10). As shown, each respective dial indicator surface 171 can have a unique height to distinguish itself from other dial indicator surfaces 171; when visually compared with the reference indicators 130 a practitioner can determine which “pressure setting” the valve unit 100 is at.

FIGS. 8B-8K show various shapes of dial indicator surfaces 171 that can be distinguished under cross-sectional imaging (as demonstrated in FIGS. 7A and 7B discussed above). FIG. 8B shows a simple “solid block” shape, FIG. 8C shows a narrow triangular notch shape, FIG. 8D shows a narrow semicircular notch shape, and FIG. 8E shows a single squared notch shape. FIG. 8F shows a wide triangular notch shape, FIG. 8G shoes a wide semicircular notch shape, and FIG. 8H shows a double squared notch shape. FIG. 8I shows an asymmetric notch shape (that may also help with distinguishing an orientation of the device as well), FIG. 8J shows a semicircular protrusion shape, and FIG. 8K shows a triangular protrusion shape. The dial indicator surfaces 171 and reference indicator surfaces 131 can be of any suitable shape, size, and general configuration including but not limited to the examples shown in FIGS. 2A and 4A-8K.

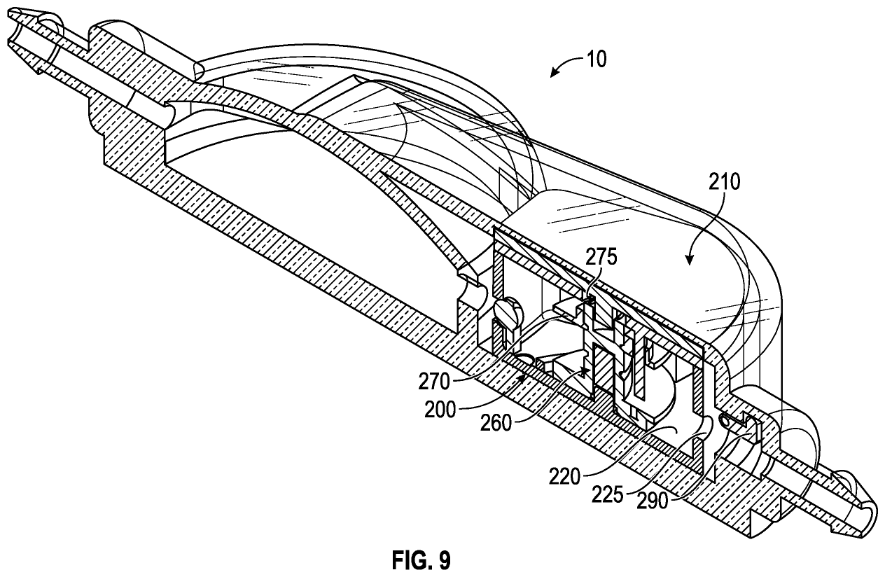

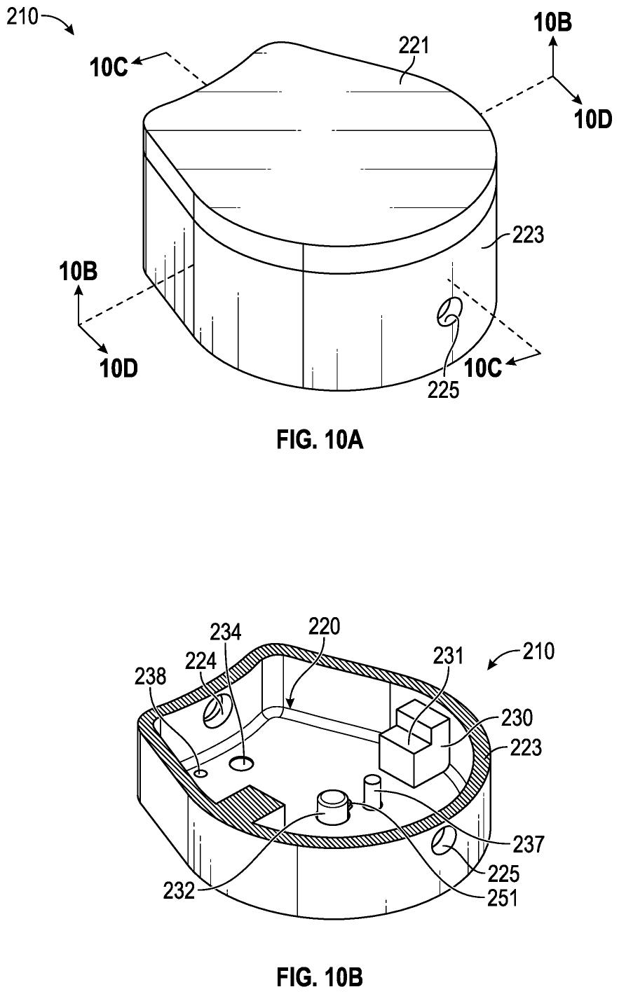

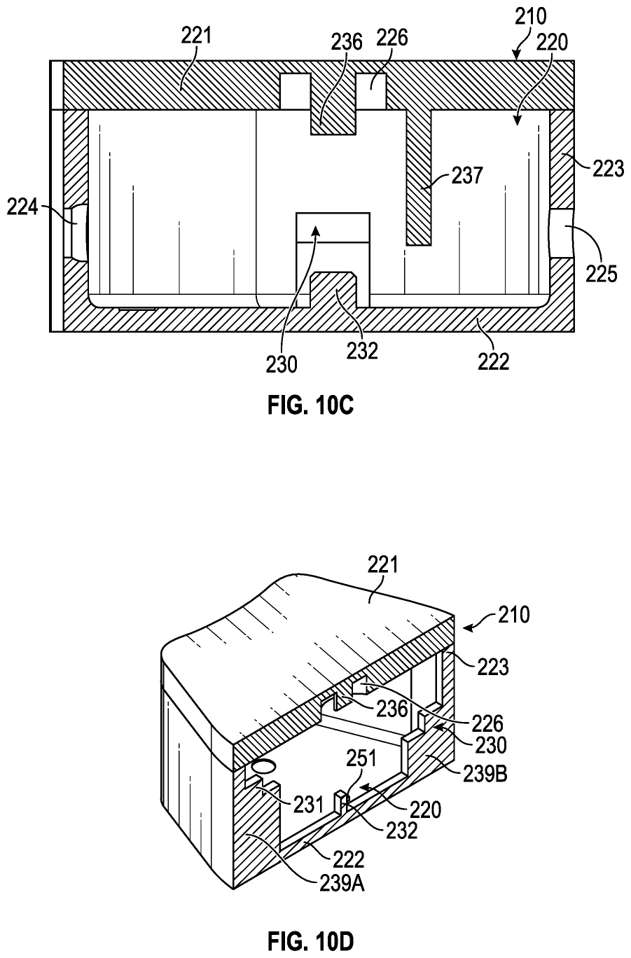

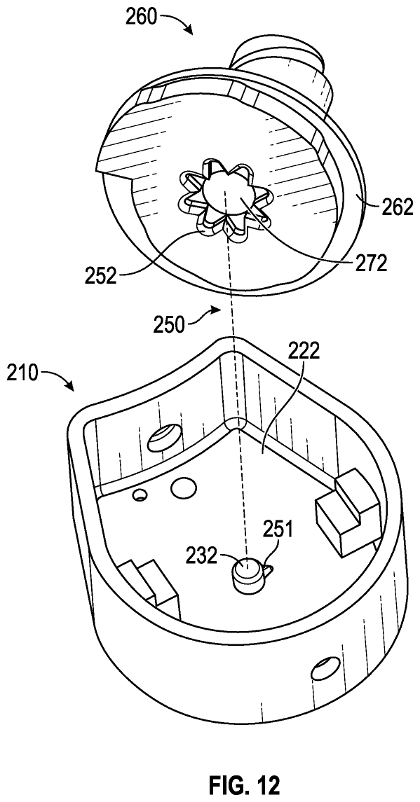

FIG. 9 shows a cross-sectional view of a second embodiment of a valve unit 200 of the valve device 10 of FIGS. 1A and 1B. As shown, a housing 210 of the valve unit 200 includes a cavity 220 that receives a dial 260 therein for controlling a flow of material through the valve unit 200 and for indicating a “pressure setting” of the valve unit 200 when observed through non-invasive cross-sectional imaging techniques such as ultrasound. The dial 260 is moveable within the housing 210 between a plurality of positions; the position of the dial 260 corresponds with the “pressure setting” of the valve unit 200. The dial 260 includes a dial indicator 270 that indicates the position of the dial 260 (and therefore the “pressure setting” of the valve unit 200) and can be observed through cross-sectional imaging. The housing 210 includes one or more reference indicators 230 (FIG. 10D) that can aid a practitioner in determining an orientation of the valve unit 200 and determining a position of the dial indicator 270 to ensure correct interpretation of the “pressure setting” of the valve unit 200. As further shown, and as will be discussed in greater detail below, the valve unit 200 can include a locking mechanism 250 (FIG. 12) having or otherwise associated with a tensioning element 275 that prevents unintentional alteration of the position of the dial 260. FIG. 9 further shows an outlet 225 of the valve unit 200 and a flow indicator 290 that indicates flow of material through the valve unit 200 when observed through non-invasive cross-sectional imaging techniques such as ultrasound.

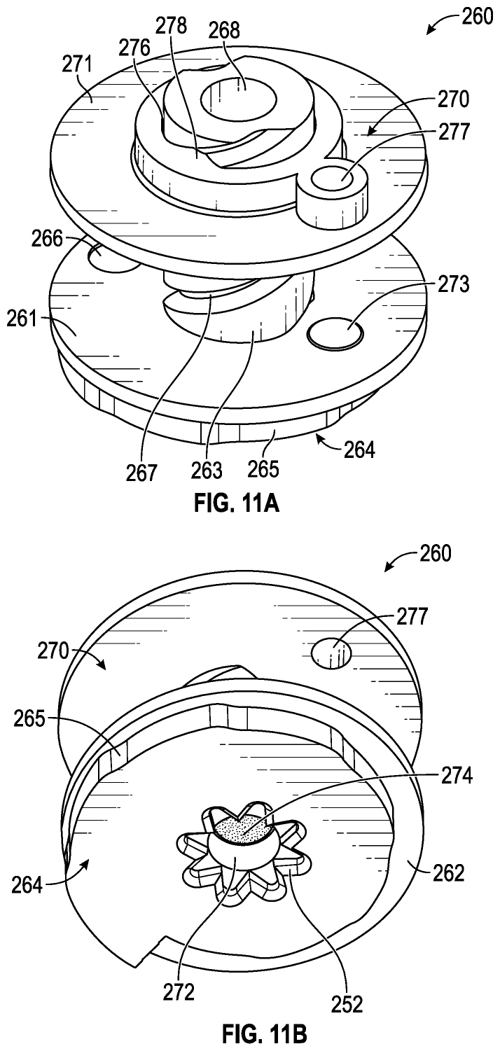

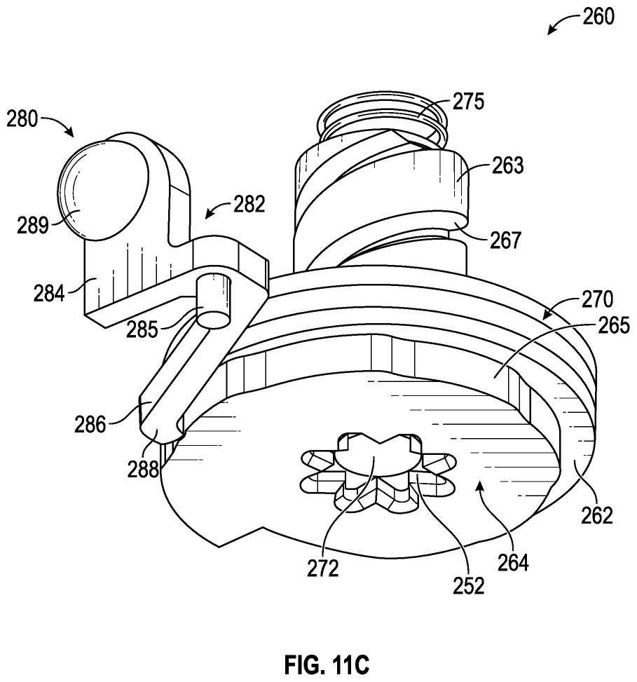

FIGS. 10A-10D illustrate the housing 210 of the valve unit 200 that receives the dial 260 (FIG. 9) therein. As shown, the housing 210 can include a top surface 221, a bottom surface 222 defined opposite from the top surface 221, and sidewalls 223 between the top surface 221 and the bottom surface 222 that define the outlet 225. The housing 210 further defines an inlet 224 opposite from the outlet 225; in operation, material enters the housing 210 at the inlet 224 and exits the housing 210 at the outlet 225. In some embodiments, the housing 210 can include one or more radiopaque elements 234 that enable aspects of the valve unit 200 to be detectable under x-ray in addition to cross-sectional imaging. FIGS. 10B-10D show the housing 210 having the reference indicators 230; in particular, the reference indicators 230 can include a plurality of reference indicator surfaces 231 that can be configured in a stepped arrangement as shown. In the embodiment shown, the reference indicators 230 can include a first reference indicator grouping 239A and a second reference indicator grouping 239B along opposing sides of the cavity 220 that each include reference indicator surfaces 231 at different heights to help indicate an orientation of the valve unit 200. When viewed in cross-section from a side of the housing 210, the varying vertical positions of each reference indicator surface 231 are clearly visible and remain stationary relative to the housing 210; this aspect enables practitioners to use the reference indicators 230 as reference to determine a position of the dial 260, and thereby the “pressure setting” enforced by the dial 260. The housing 210 can also include a dial stem 232 that receives the dial 260 in an arrangement that will be discussed in greater detail below; in the example shown, the housing 210 can further include a locking mechanism protrusion 251 of the locking mechanism 250 (FIG. 12) along the dial stem 232 that engages the dial 260 in an arrangement that will be discussed in greater detail below. In a further aspect, with reference to FIG. 10B, the housing 210 can include a flow lever receptacle 238 positioned near the inlet 224 for coupling with a flow lever 282 (FIG. 11C) in an arrangement that will be discussed in greater detail below. The housing 210 can further include a top surface recess 226 having a dial guide element 236 protruding therefrom for capture of the dial 260, and a disc guide element 237 that extends from the top surface 221 to engage the dial indicator 270 in an arrangement which will be described in greater detail herein.

FIGS. 11A-11C show the dial 260 of the valve unit 200 that can be encapsulated within the housing 210 as discussed above and as shown in the cross-sectional image of FIG. 9. The dial 260 is moveable between the plurality of positions within the housing 210 and controls the flow of material through the valve unit 200; in particular, the dial 260 can be rotated between the plurality of positions to select a “pressure setting” for the flow of material though the valve unit 200. As shown, the dial 260 includes a top surface 261, a bottom surface 262 defined opposite the top surface 261. The dial 260 further includes a dial aperture 272 that receives the dial stem 232 of the housing 210 when the dial 260 is coupled with the housing.

The dial 260 can include a lead screw 263 protruding outward from the top surface 261; as shown, the lead screw 263 includes a lead screw groove 267 that engages a dial indicator 270 of the dial 260. The lead screw 263 can include a lead screw aperture 268 that engages the dial guide element 236 of the housing 210 at a top of the lead screw 263. The dial indicator 270, in contrast to the dial indicator 170 of FIGS. 11A-11C, can be a disc-shaped element that engages the lead screw 263 and is operable for translation in the vertical direction along the lead screw 263 when the dial 260 rotates between different positions (e.g., between different angular positions). The dial indicator 270 includes a dial indicator surface 271 that is visible under cross-sectional imaging. As such, the dial indicator 270 indicates a “pressure setting” of the valve unit 200 by its vertical position along the lead screw 263 when viewed through cross-sectional imaging. As shown, the dial indicator 270 can include a first indicator aperture 276 through a center of the dial indicator 270 having a dial follower 278 that engages the lead screw 263. The dial indicator 270 can also include a second indicator aperture 277 adjacent to the first indicator aperture 276 that engages the disc guide element 237 of the housing 210 and ensures that the dial indicator 270 reliably moves in the vertical direction when the dial 260 rotates.

FIG. 11B shows the bottom surface 262 of the dial 260. As shown, the bottom surface 262 includes a plurality of locking mechanism recesses 252 of the locking mechanism 250 (FIG. 12) that can each receive the locking mechanism protrusion 251 (FIGS. 10B and 10C) at the dial stem 232 of the housing 210. In a further aspect, the bottom surface 262 of the dial 260 includes a cam 264 having a plurality of cam surfaces 265 as shown. With additional reference to FIG. 11C, the cam 264 of the dial 260 engages a flow control unit 280 that controls a flow of material that enters the housing 210 through the inlet 224 (FIGS. 10B and 10C). In the example shown, each cam surface 265 of the plurality of cam surfaces 265 of the dial 260 corresponds with a respective locking mechanism recess 252 of the plurality of locking mechanism recesses 252, which in turn correspond with an angular position of the dial 260. The flow control unit 280 can include the flow lever 282 in operative association with a cam follower 288 that contacts a cam surface 265 of the plurality of cam surfaces 265 as shown. The flow lever 282 can also include a flow occluder 289 positioned opposite from the cam follower 288 that contacts the inlet 224 of the housing 210 at selective pressure settings to control the flow of material through the inlet 224. In the embodiment shown, the cam surface 265 occupied by the cam follower 288 determines a pressure applied at the inlet 224 by the flow occluder 289; as such, the cam surface 265 occupied by the cam follower 288 corresponds directly with the position of the dial 260. In some embodiments, the flow lever 282 can include a first lever arm 284 and a second lever arm 286 that couple at a hinge 285 that inserts into the flow lever receptacle 238 (FIG. 10B) of the housing 210. Further, as shown, the cam 264 can include a “minimum flow” cam surface 265A and a “maximum flow” cam surface 265B. When the cam follower 288 occupies the minimum flow cam surface 265A, the pressure applied at the inlet 224 by the flow occluder 289 is minimized and the dial indicator 270 is at a maximum height along the lead screw 263; conversely, when the cam follower 288 occupies the maximum flow cam surface 265B, the pressure applied at the inlet 224 by the flow occluder 289 is maximized and the dial indicator 270 is at a minimum height along the lead screw 263.

In the example shown in FIGS. 11A-11C, the dial 260 is configured for eight unique “pressure settings”, dictated by the plurality of cam surfaces 265 and the plurality of locking mechanism recesses 252 that correspond with respective cam surfaces 265. The vertical position of the dial indicator 270 relative to the housing 210 (including the reference indicator surfaces 231 of the housing 210) can inform practitioners as to the position of the dial 260 and thereby the “pressure setting” of the valve unit 200. Note that while eight unique dial indicator surfaces 271 are shown in FIGS. 11A-11C, other embodiments are contemplated and shown herein that include more or fewer unique dial indicator surfaces 271 to corresponding “pressure settings”. In a further aspect, the dial 260 can also include a radiopaque element 266 that enables a practitioner to determine a position of the dial 260 through x-ray imaging if necessary.

As shown, the dial 260 can include a magnetic-responsive element (e.g., a first magnetic-responsive element 273 and a second magnetic-responsive element 274) that, when influenced by an externally-applied magnetic force by the external magnetic device 90, enables alteration of the position of the dial 260. The first magnetic-responsive element 273 can be positioned adjacent to the lead screw 263 for rotating the dial 260; the second magnetic-responsive element 274 can be positioned within the lead screw 263, particularly within the dial aperture 272, for disengaging the locking mechanism 250 and lifting the dial 260 away from the bottom surface 222 of the housing 210 such that the dial 260 can subsequently be rotated using the first magnetic-responsive element 273 and the external magnetic device 90 in an arrangement which will be discussed in greater detail herein. In particular, when influenced by an external magnetic device 90, the first magnetic-responsive element 273 and the second magnetic-responsive element 274 can be attracted to or repulsed by the external magnetic device 90 and can thus be used to rotate or otherwise move the dial 260 to a desired position of the plurality of positions of the dial 260. Rotation of the dial 260 to a new position will also cause the cam follower 288 of the flow control unit 280 to occupy a new cam surface 265 of the cam 264 of the dial 260, which changes the “pressure setting” of the valve unit 200.

FIG. 12 shows the engagement of the dial 260 with the housing 210 at a locking mechanism 250 of the valve unit 200. As discussed, the housing 210 can include the locking mechanism protrusion 251 of the locking mechanism 250 along the bottom surface 222 of the housing 210 and surrounding the dial stem 232. The housing 210 is shown in FIG. 12 with the top surface 221 removed. The dial 260 can include the plurality of locking mechanism recesses 252 of the locking mechanism 250 along the bottom surface 262 of the dial 260 and surrounding the dial aperture 272. As illustrated, the dial 260 couples with the housing 210 by insertion of the dial stem 232 of the housing 210 into the dial aperture 272 of the dial 260. One locking mechanism recess 252 of the plurality of locking mechanism recesses 252 along the dial 260 engage the locking mechanism protrusion 251 along the dial stem 232 of the housing 210 as shown. When the second magnetic-responsive element 274 is not being activated by the external magnetic device 90 (e.g., absent external application of the magnetic force by the external magnetic device 90), the bottom surface 262 of the dial 260 rests against the bottom surface 222 of the housing 210 and the locking mechanism protrusion 251 couples with the locking mechanism recess 252 to prevent rotation of the dial 260 within the housing 210. When the second magnetic-responsive element 274 is being activated by the external magnetic device 90, the bottom surface 262 of the dial 260 lifts up from the bottom surface 222 of the housing 210 to deactivate the locking mechanism 250 such that the locking mechanism protrusion 251 decouples from the locking mechanism recess 252 to allow rotation of the dial 260 within the housing 210. With the locking mechanism 250 deactivated, the dial 260 can be rotated to a new position by the external magnetic device 90 interacting with the first magnetic-responsive element 273.

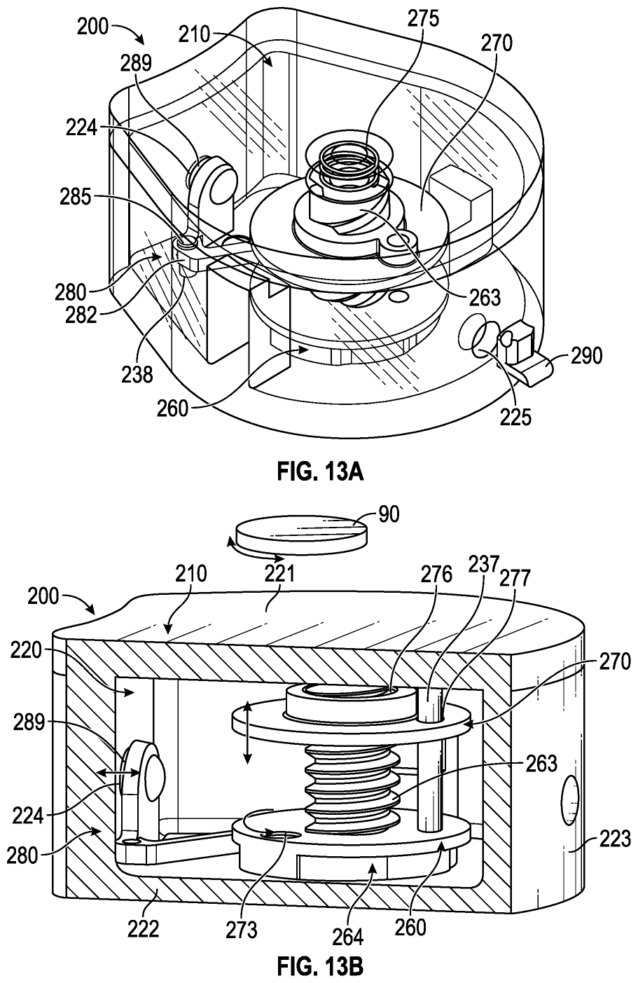

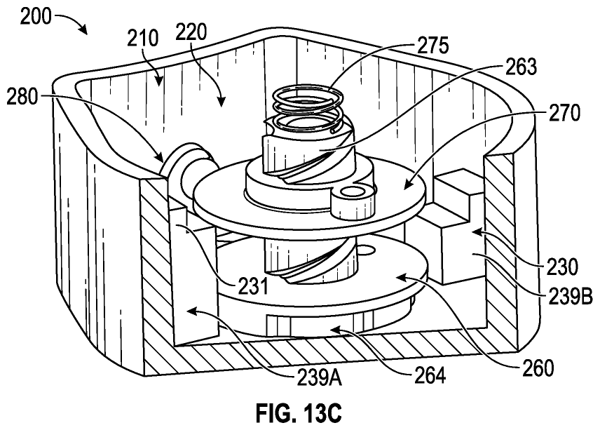

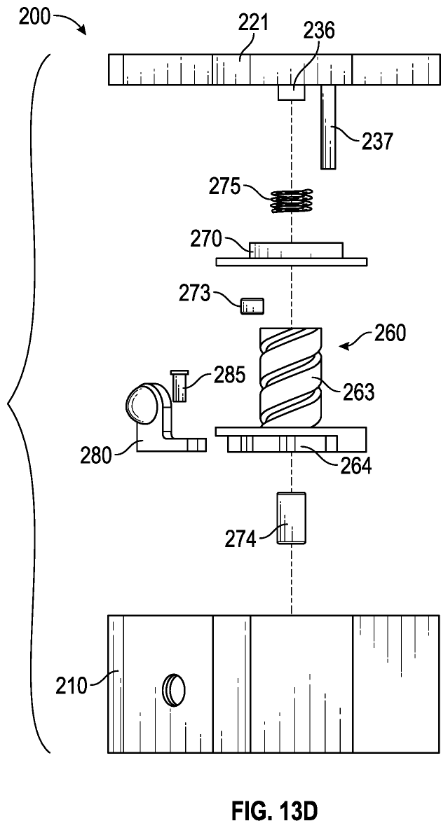

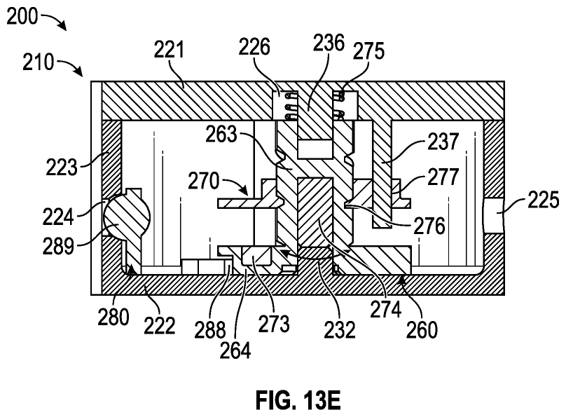

FIGS. 13A-13E show the valve unit 200 including the dial 260, the flow control unit 280 and the housing 210, with FIG. 13D showing an exploded view of the valve unit 200. As discussed, the dial aperture 272 of the dial 260 receives the dial stem 232 of the housing 210 at a bottom of the dial 260; likewise, the lead screw aperture 268 of the lead screw 263 receives the dial guide element 236 at a top of the lead screw 263 as shown. The dial indicator 270 receives the lead screw 263 of the dial 260 at the first indicator aperture 276 of the dial indicator 270 and receives the disc guide element 237 of the housing 210 at the second indicator aperture 277 as shown. The cam follower 288 of the flow control unit 280 contacts the cam 264 of the dial 260 to selectively control a flow of material through the valve unit 200; the “pressure setting” of the valve unit 200 depends on which cam surface 265 of the cam 264 is occupied by the cam follower 288 of the flow control unit 280. The hinge 285 of the flow lever 282 of the flow control unit 280 couples with the flow lever receptacle 238 of the housing 210 as shown. In some embodiments, the flow lever 282 can include the flow occluder 289 that contacts the inlet 224 of the housing 210 at variable pressure (e.g., dependent upon which cam surface 265 of the cam 264 is occupied by the cam follower 288) to control the flow of material through the valve unit 200. As further shown, the dial 260 can include the tensioning element 275 positioned between the lead screw 263 of the dial 260 and the top surface 221 of the housing 210; in the embodiment shown, the tensioning element 275 can be positioned within the top surface recess 226 of the housing 210, where the dial guide element 236 extends from the top surface recess 226 as shown in FIG. 13E. The tensioning element 275 ensures that the bottom surface 262 of the dial 260 rests against the bottom surface 222 of the housing 210 and the locking mechanism protrusion 251 couples with a locking mechanism recess 252 of the plurality of locking mechanism recesses 252 to prevent rotation of the dial 260 within the housing 210 when the second magnetic-responsive element 274 is not being activated by the external magnetic device 90. When the second magnetic-responsive element 274 is being activated by the external magnetic device 90, the tensioning element 275 can compress to enable the bottom surface 262 of the dial 260 to lift up from the bottom surface 222 of the housing 210 and deactivate the locking mechanism 250 such that the locking mechanism protrusion 251 decouples from the locking mechanism recess 252. To rotate the dial 260 and alter the setting of the dial 260, a practitioner can lift the dial 260 away from the bottom surface 222 of the housing 210 by the external magnetic device 90 (which interacts with the first magnetic-responsive element 273 and the second magnetic-responsive element 274) and rotate the external magnetic device 90 in a clockwise or counterclockwise direction such that the first magnetic-responsive element 273 follows the external magnetic device 90, thereby rotating the dial 260 (including the lead screw 263) in the clockwise or counterclockwise direction. This motion likewise causes the flow occluder 289 of the flow control unit 280 to adjust a pressure being applied at the inlet 224 of the housing 210.

Further, the flow indicator 290 is shown positioned distal to the outlet 225 of the housing 210. FIG. 13E shows a cross-sectional side view of the housing 210 and the dial 260. The dial 260 is shown in a position of the plurality of positions of the dial 260 identifiable by the position the dial indicator surface 271 of the dial indicator 270 as shown. The vertical position of the dial indicator surface 271 is visible under cross-sectional imaging.

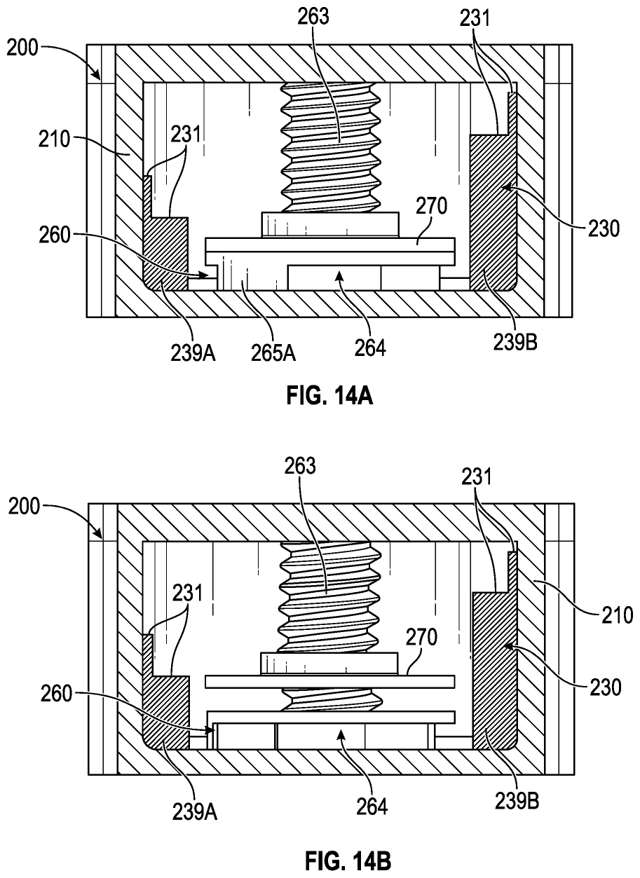

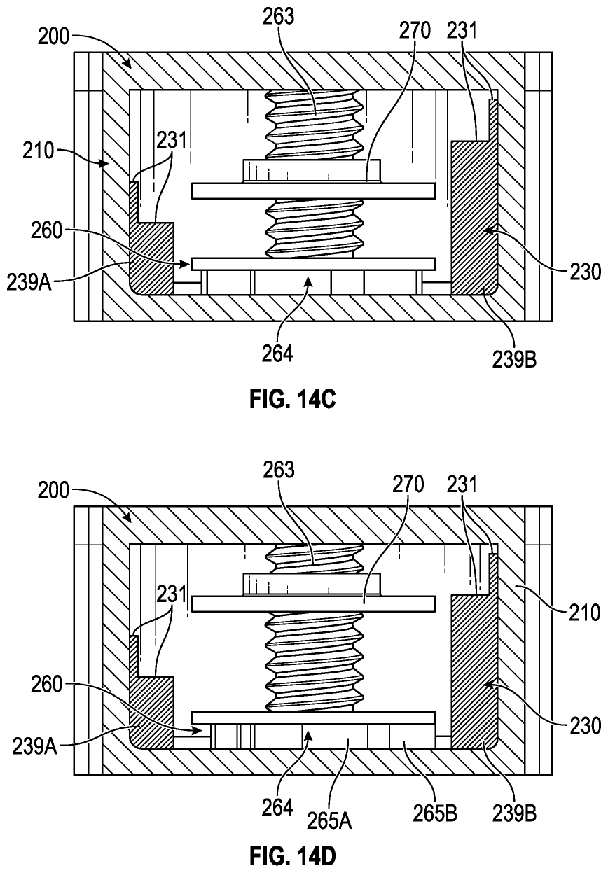

FIGS. 14A-14D show a series of cross-sectional side views of the valve unit 200, particularly the dial indicator 270 and the reference indicators 230. In the examples shown, the reference indicators 230 include the first reference indicator grouping 239A and the second reference indicator grouping 239B along opposing sides of the cavity 220 that each include reference indicator surfaces 231 at different heights to help indicate an orientation of the valve unit 200. FIG. 14A shows a first “pressure setting” of the valve unit 200 indicated by a first vertical position of the dial indicator 270 along the lead screw 263, where the reference indicators 230 along the sides of the cavity 220 can provide reference to a practitioner to help discern the vertical position of the dial indicator 270; note that the cam follower (not shown) would occupy the minimum flow cam surface 265A along the cam 264 of the dial 260 visible in FIG. 14A. FIG. 14B shows a second “pressure setting” of the valve unit 200 indicated by a second vertical position of the dial indicator 270 along the lead screw 263; note that the cam follower (not shown) would occupy another cam surface 265. FIG. 14C shows a fourth “pressure setting” of the valve unit 200 indicated by a fourth vertical position of the dial indicator 270 along the lead screw 263, and FIG. 14D shows a sixth “pressure setting” of the valve unit 200 indicated by a sixth vertical position of the dial indicator 270 along the lead screw 263.

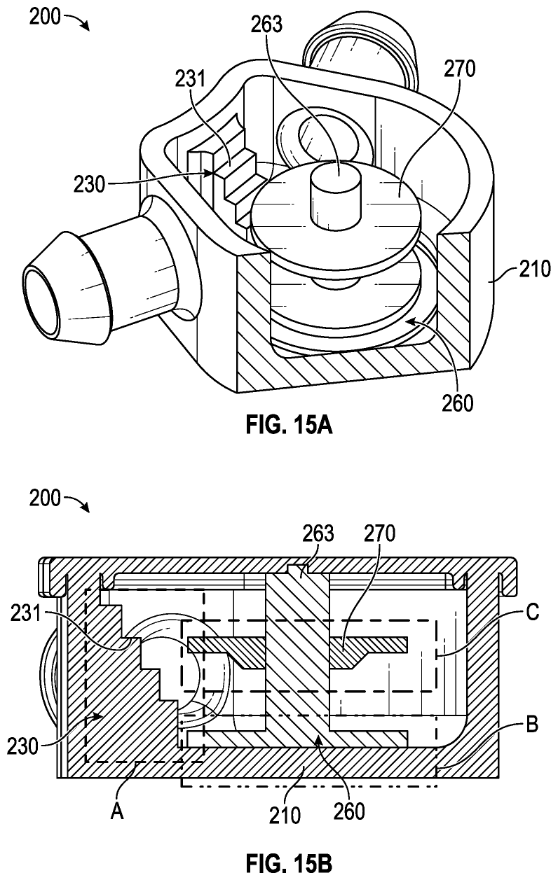

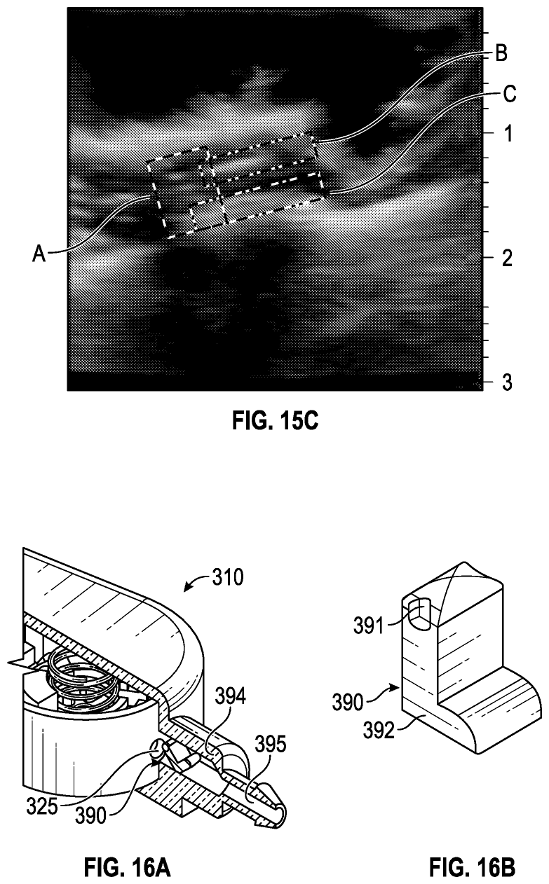

FIGS. 15A-15C shows an alternate arrangement of the valve unit 200 of FIGS. 9-14D where reference indicators 230 are grouped in a single grouping but still show the plurality of reference indicator surfaces 231 at different heights to help indicate an orientation of the valve unit 200. The dial 260 includes the dial indicator 270 at a vertical position along the lead screw 263 as shown. FIG. 15B shows a cross-sectional view of the housing 210 and the dial 260 of FIG. 15A, which can be compared directly with FIG. 15C which shows a corresponding cross-sectional image of the housing 210 and the dial 260 captured by ultrasound. The reference indicators 230 are indicated by Box A of FIGS. 15B and 15C, the dial indicator 270 is indicated by Box B of FIGS. 15B and 15C, and the dial 260 is indicated by Box C of FIGS. 15B and 15C. A practitioner can determine the position of the dial indicator 270, and thereby the “pressure setting” of the valve unit 200 by identifying relative positions of the reference indicators 230, the dial indicator 270 and dial 260 within the cross-sectional image captured by ultrasound. The practitioner can determine an orientation of the valve unit 200 by observing which side the reference indicators 230 are on and can then determine the “pressure setting” of the valve unit 200 by comparing the vertical position of the dial indicator 270 relative to the reference indicators 230.

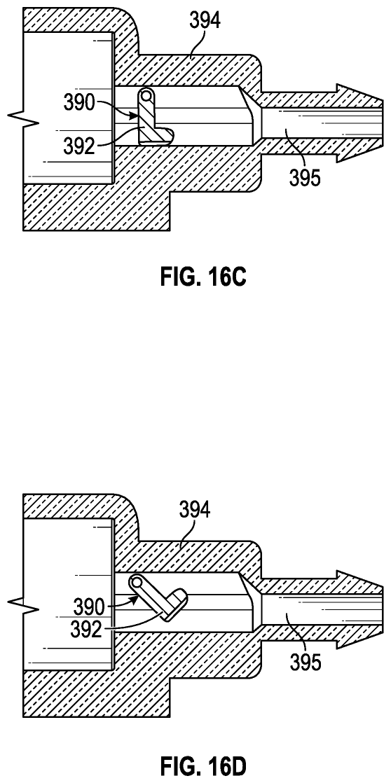

FIGS. 16A-16D show a first embodiment of the flow indicator 390 positioned distal to an outlet 325 of a housing 310. The flow indicator 390 can be positioned within a flow indicator chamber 394 that communicates with or otherwise forms a part of a distal lumen 395 that communicates with the outlet 325 of the housing 310. The flow indicator 390 can similarly be viewed through cross-sectional imaging to indicate if material is flowing out of the housing 310. The flow indicator 390 can include a flow indicator hinge 391 that couples within the flow indicator chamber 394 and enables the flow indicator 390 to rotate about the flow indicator hinge 391 when material is flowing through the distal lumen 395. As shown, the flow indicator 390 can include a flow indicator bend 392 that aids practitioners in identifying the flow indicator 390 within cross-sectional imaging. FIG. 16C shows one example arrangement of the flow indicator 390 positioned within the distal lumen 395 when no material is flowing through the distal lumen 395; in this example, the flow indicator bend 392 sits “perpendicularly” within the flow indicator chamber 394 as shown. Conversely, FIG. 16D shows the flow indicator 390 of FIG. 16C when material is flowing through the distal lumen 395; in this example, the flow indicator bend 392 is pushed outward by the material to an angled position within the flow indicator chamber 394 as shown.

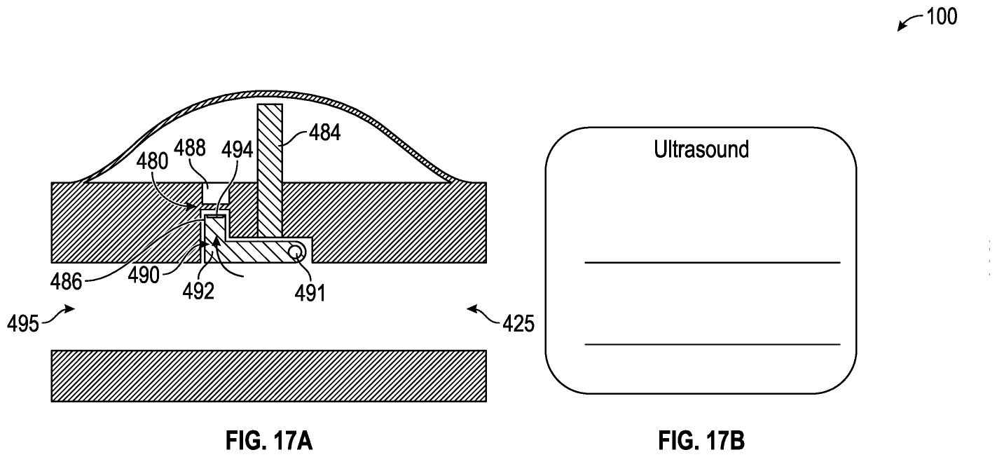

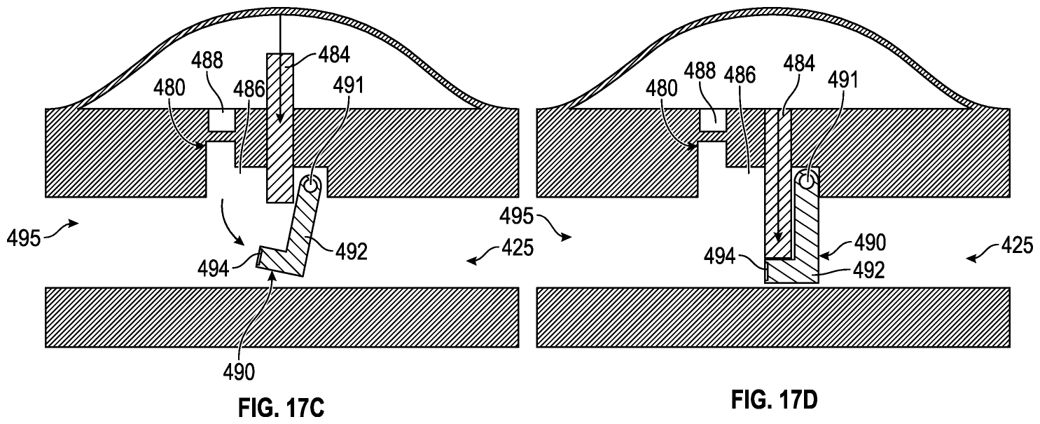

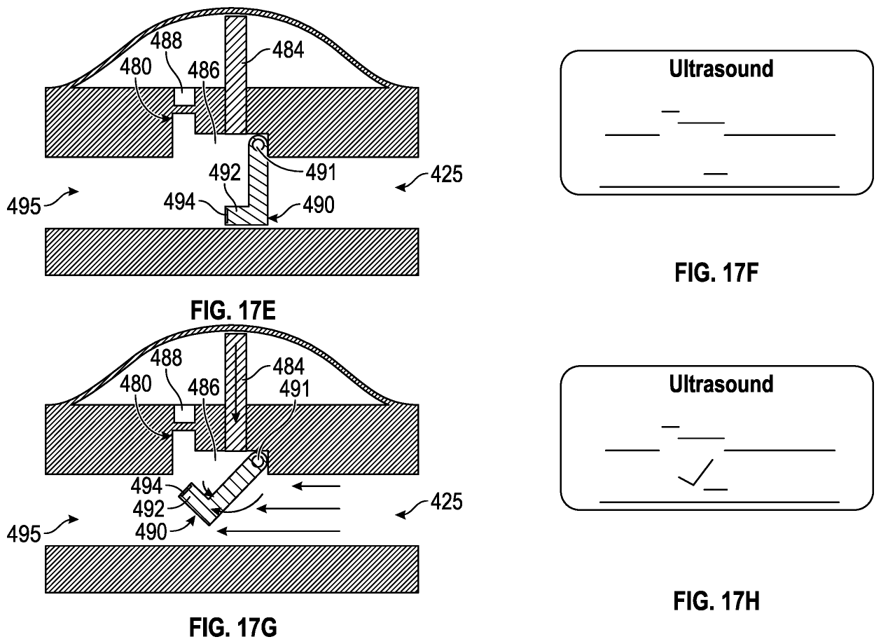

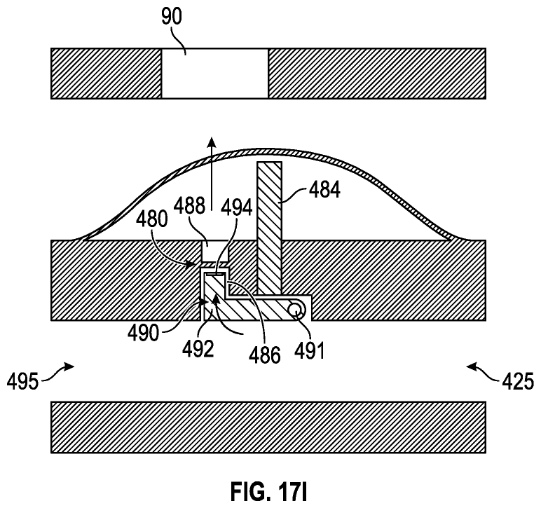

FIGS. 17A-171 show a second embodiment of the flow indicator 490 positioned distal to an outlet 425 of a housing (not shown, but analogous to housing 110 or 210 of FIGS. 1A-15C). The flow indicator 490 can be positioned within a flow indicator chamber 480 that communicates with or otherwise forms a part of a distal lumen 495 that communicates with the outlet 425 of the housing. The flow indicator 490 can similarly be viewed through cross-sectional imaging to indicate if material is flowing out of the housing. The flow indicator chamber 480 can include a flow indicator seat 486 positioned above the distal lumen 495 such that when the flow indicator 490 is not being interrogated, the flow indicator 490 can rest within the flow indicator seat 486 without occluding the flow of material through the distal lumen 495. In this arrangement, the flow indicator seat 486 can include a first magnetic element 488 that magnetically interacts with a flow indicator capture element 494 (which can be magnetic-responsive) of the flow indicator 490 to retain the flow indicator 490 within the flow indicator seat 486 as shown in FIG. 17A when not being interrogated. FIG. 17B shows an example diagram of how the flow indicator 490 would appear in a corresponding cross-sectional image when in the arrangement of FIG. 17A. As shown, the flow indicator 490 can similarly include a flow indicator bend 492 that aids practitioners in identifying the flow indicator 490 within cross-sectional imaging. The flow indicator 490 can include a flow indicator hinge 491 that couples within the flow indicator chamber 480 and enables the flow indicator 490 to rotate about the flow indicator hinge 491 when material is flowing through the distal lumen 495 and/or following manual actuation for interrogation.

The flow indicator chamber 480 can further include a flow indicator actuator 484 that, when manually actuated, causes the flow indicator 490 to rotate from the “non-occluding” position shown in FIG. 17A to an “intermediate” position shown in FIG. 17C, and finally to an “interrogating” position shown in FIG. 17D. Once the flow indicator 490 is in the “interrogating” position shown in FIG. 17D, the flow indicator actuator 484 can return to a default position as shown in FIG. 17E (this may be accomplished by a tensioning element, not shown). FIG. 17F shows an example diagram of how the flow indicator 490 would appear in a corresponding cross-sectional image when in the arrangement of FIG. 17E. When no material is flowing through the distal lumen 495, the flow indicator bend 492 sits “perpendicularly” within the flow indicator chamber 480. FIG. 17G shows the flow indicator 490 of FIG. 17E when material is flowing through the distal lumen 495; in this example, the flow indicator bend 492 is pushed by the material to an angled position within the flow indicator chamber 480 as shown. FIG. 17H shows an example diagram of how the flow indicator 490 would appear in a corresponding cross-sectional image when in the arrangement of FIG. 17G. To return the flow indicator 490 to the “non-occluding” position shown in FIG. 17A, an external magnetic device 90 can activate the first magnetic element 488 and/or the flow indicator capture element 494 of the flow indicator 490 to draw the flow indicator capture element 494 and thereby the flow indicator 490 away from the distal lumen 495 and into the flow indicator seat 486 as shown in FIG. 17I.

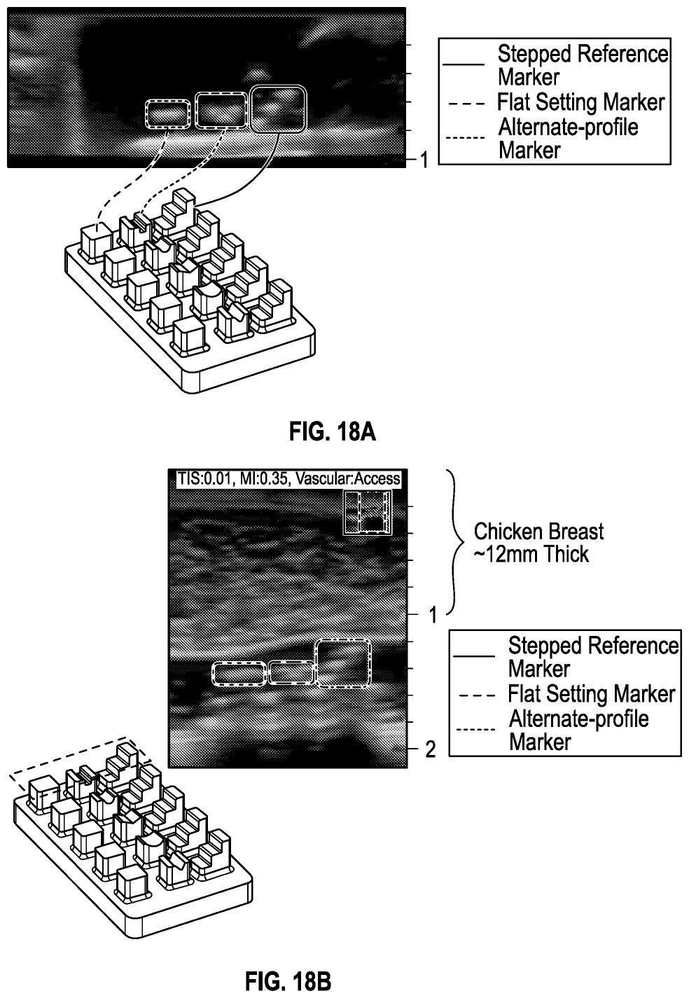

FIGS. 18A and 18B show various testing arrangements that were used to determine how well the indicators appear in captured cross-sectional images, especially reference indicators 230 and dial indicator 270 of FIGS. 2A-8K. FIG. 18B in particular shows how these indicators appear underneath chicken breast (simulating scalp tissue). As shown, the stepped reference marker (corresponding with reference indicators 230 of FIGS. 2A-15C), the flat setting marker, and the “alternate-profile” marker are well-distinguishable when viewed through non-invasive cross-sectional imaging.

The present disclosure provides further discussion on material properties that are contemplated for various components of the valve device 10 and associated valve units 100 or 200 outlined herein.

In one aspect, an outer covering of the valve device 10 can be of a biocompatible and flexible material with low acoustic attenuation (e.g., silicone rubber or another suitable material). The housing 110 (210) of the valve unit 100 (200) can be of a biocompatible and rigid material with low acoustic attenuation (e.g., polymers such as polyethylene or another suitable material). The reference indicators 130 (230) and the dial indicator(s) 170 (270) can be of a biocompatible and non-ferrous (or otherwise magnetically unresponsive) material with clear visibility under ultrasound imaging; for example, the reference indicators 130 (230) and the dial indicator(s) 170 (270) can be of a material with high acoustic impedance, e.g., ceramics, silicon carbide, polymers such as Delrin, HDPE, or another suitable polymer, non-ferrous metals such as titanium, gold, aluminum, or another suitable metal or metal alloy, crystals such as sapphire, ruby, or another suitable crystalline material. Radiopaque elements 134, 135, 166, 234 and/or 266 can be of a biocompatible, ferrous or non-ferrous material with clear visibility under x-ray imaging, e.g., high density substances such as barium sulfate, bismuth compounds, metals or metal-alloys such as tungsten, titanium, zinc-magnesium, cobalt-chromium, or another suitable metal or metal-alloy. Alternatively, these materials can also be used as fillers within polymer materials herein. Flow indicator 190, 290, 390 or 490 can be of a biocompatible material having a non-ferrous (or otherwise magnetically unresponsive) body with clear visibility under ultrasound imaging (such as a material having high acoustic impedance). Flow indicator capture element 494 can include a ferrous or otherwise magnetically responsive material that can be coated in a biocompatible layer. Flow indicator actuator 484 be of a biocompatible and rigid material with low acoustic attenuation. Tensioning element 175 (275) can of a biocompatible and flexible material having a small spring constant, minimal ultrasound artifact with acoustic impedance that can be similar to that of soft tissue such as cerebrospinal fluid or another biofluid (e.g., ˜1.5MRayls). One example material can be a polymer such as polyethylene, or can include metals such as spring steel, titanium, nitinol.

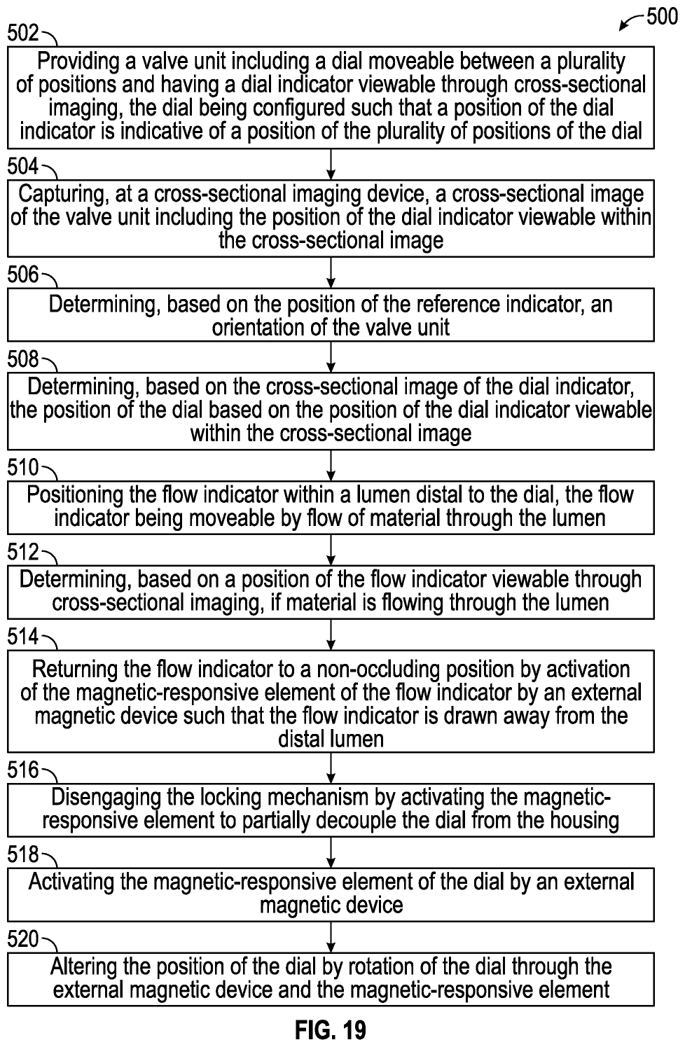

FIG. 19 shows a method 500 for controlling and interrogating a flow of material through a valve unit.

Step 502 of method 500 includes providing a valve unit (e.g., valve unit 100 or 200), the valve unit including a dial moveable between a plurality of positions and having a dial indicator viewable through cross-sectional imaging, the dial being configured such that a position of the dial indicator is indicative of a position of the plurality of positions of the dial. Step 504 of method 500 includes capturing, at a cross-sectional imaging device, a cross-sectional image of the valve unit including the position of the dial indicator viewable within the cross-sectional image. Step 506 of method 500 includes determining, based on the position of the reference indicator, an orientation of the valve unit. Step 508 of method 500 includes determining, based on the cross-sectional image of the dial indicator, the position of the dial based on the position of the dial indicator viewable within the cross-sectional image.

To interrogate the valve unit to see if material is flowing through the lumen: Step 510 of method 500 includes positioning the flow indicator within a lumen (e.g., distal lumen 495) distal to the dial, the flow indicator being moveable by flow of material through the lumen. Step 512 of method 500 includes determining, based on a position of the flow indicator viewable through cross-sectional imaging, if material is flowing through the lumen. Step 514 of method 500 includes returning the flow indicator to a non-occluding position by activation of the magnetic-responsive element of the flow indicator by an external magnetic device such that the flow indicator is drawn away from the distal lumen.

To configure or otherwise select a “pressure setting” for the valve unit: Step 516 of method 500 includes disengaging the locking mechanism by activating the magnetic-responsive element to partially decouple the dial from the housing. Step 518 of method 500 includes activating the magnetic-responsive element of the dial by an external magnetic device. Step 520 of method 500 includes altering the position of the dial by rotation of the dial through the external magnetic device and the magnetic-responsive element.

Further, the valve device 10 (including associated valve unit 100 or 200) can be applied for treatment of various diseases and/or conditions including but not limited to normal pressure hydrocephalus, obstructive hydrocephalus, pseudo tumor cerebri, congenital ventriculomegaly, chronic arachnoid cyst, and/or chronic and complex cerebrospinal fluid leak. In one aspect, the valve device 10 can be implanted within the body in communication with a medical tube for controlling a flow of material through the medical tube. The valve device 10 can be implanted for treatment of hydrocephalus or another similar condition requiring a ventriculoperitoneal shunt (e.g., from a ventricle of the brain to the peritoneal cavity), and where the valve device 10 can communicate with or otherwise include a ventriculoperitoneal shunt catheter positioned between the peritoneal cavity of the body and a ventricle of the brain. With respect to other diseases and/or conditions mentioned above, the valve device 10 can communicate with or otherwise include a ventriculopleural shunt catheter positioned between a pleural cavity of the body and a ventricle of the brain. The valve device 10 can include or otherwise communicate with a ventriculoatrial shunt catheter positioned between an atrium of a heart of the body and a ventricle of the brain. The valve device 10 can include or otherwise communicate with a cisternoperitoneal shunt catheter positioned between a peritoneal cavity of the body and a subarachnoid cistern of the brain. The valve device 10 can include or otherwise communicate with a cisternopleural shunt catheter, positioned between the pleural cavity of the body and a subarachnoid cistern of the brain. The valve device 10 can include or otherwise communicate with a cisternoatrial shunt catheter positioned between an atrium of a heart of the body and a subarachnoid cistern of the brain. In another aspect, valve device 10 can be employed and installed according to various embodiments and methods described herein for diversion of fluid from a cavity of the body at a first location of the body to an external environment at a second location of the body or for communicating fluid from an external environment at the first location to a cavity of the body at the second location. The valve device 10 can be controlled and interrogated as discussed above with reference to method 500 to ensure proper function when the valve device 10 is employed during treatment of any of the above diseases and/or conditions. As readily apparent to one of skill in the art, any number of conditions and disease treatment regimens associated with shunt implantation and evaluation, as well as diagnostic or prognostic regimens associated with shunt implantation and evaluation, could be used in conjunction with the various embodiments of valve device 10,

In some embodiments, the valve device 10 can be offered as a kit that includes the valve unit 100 or 200, the flow indicator 190, 290, 390 or 490, and further including the external magnetic device 90 for adjustment of the valve unit 100 or 200 and interrogation of the flow indicator 190, 290, 390 or 490. The kit may further include any medical tubes or catheters that may be used in conjunction with the valve device 10, including but not limited to a ventriculoperitoneal shunt catheter, a ventriculopleural shunt catheter, a ventriculoatrial shunt catheter, a cisternoperitoneal shunt catheter, a cisternopleural shunt catheter, and a cisternoatrial shunt catheter.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A valve device, including:
a dial having a dial indicator, the dial and the dial indicator being moveable between a plurality of positions, the dial being configured such that a position of the plurality of positions of the dial is viewable through cross-sectional imaging of the dial indicator.

2. The valve device of claim 1, further comprising:
a housing encapsulating the dial and including a reference indicator, the reference indicator being viewable through cross-sectional imaging of the housing;
wherein a position of the dial indicator relative to the reference indicator is indicative of the position of the plurality of positions of the dial.

3. The valve device of claim 1, further comprising:
a flow limiter in operative association with the dial, the flow limiter being operable for limiting a flow of material through a lumen based on the position of the plurality of positions of the dial.

4. The valve device of claim 1, wherein the dial indicator includes a plurality of dial indicator surfaces identifiable through cross-sectional imaging of the dial indicator, wherein a position of each dial indicator surface of the plurality of dial indicator surfaces relative to a reference indicator is indicative of the position of the plurality of positions of the dial.

5. The valve device of claim 4, wherein each dial indicator surface of the plurality of dial indicator surfaces is identifiable through cross-sectional imaging by a vertical position and/or a cross-sectional shape of the dial indicator surface relative to the reference indicator.

6. The valve device of claim 1, wherein the dial indicator includes a dial indicator surface identifiable through cross-sectional imaging of the dial indicator, wherein a vertical position of the dial indicator surface relative to a reference indicator is indicative of the position of the plurality of positions of the dial.

7. The valve device of claim 6, wherein the dial indicator surface is moveable along a lead screw in association with the dial.

8. The valve device of claim 1, wherein the dial includes a locking mechanism that, when engaged, prevents alteration of the position of the dial.

9. The valve device of claim 8, wherein the dial includes a first magnetic-responsive element that, when activated by an external magnetic device, disengages the locking mechanism to enable alteration of the position of the dial.

10. The valve device of claim 8, wherein the dial includes a second magnetic-responsive element that, when activated by an external magnetic device, enables rotation of the dial in a clockwise direction or a counterclockwise direction to alter the position of the dial.

11. The valve device of claim 1, further comprising:
a flow indicator positionable within a lumen distal to the dial, the flow indicator being moveable by flow of material through the lumen and viewable through cross-sectional imaging of the flow indicator.

12. The valve device of claim 11, further comprising:
a flow indicator actuator in operative association with the flow indicator that, when actuated, positions the flow indicator within the lumen; and
a third magnetic-responsive element that, when activated by an external magnetic device, draws the flow indicator away from the lumen.

13. A valve device, comprising:
a dial having a dial indicator and a magnetic-responsive element, the dial being moveable between a plurality of positions;
a housing having a reference indicator; and
a locking mechanism defined by the dial and the housing;
wherein external application of a magnetic force in a vertical direction draws the dial away from the housing to disengage the locking mechanism;
wherein external application of the magnetic force enables rotation of the dial to a position of the plurality of positions by the magnetic-responsive element; and
wherein a position of the dial indicator relative to the reference indicator is observable through cross-sectional imaging and is dependent upon the position of the dial.

14. The valve device of claim 13, further comprising a tensioning element in association with the dial, the housing, and the locking mechanism such that the tensioning element returns the dial to the housing to engage the locking mechanism and prevent rotation of the dial absent the external application of the magnetic force.

15. A method, comprising:
providing a valve device including a dial moveable between a plurality of positions, the dial having a dial indicator viewable through cross-sectional imaging, the dial being configured such that a position of the dial indicator is indicative of a position of the plurality of positions of the dial;
capturing, at a cross-sectional imaging device, a cross-sectional image of the valve device including the position of the dial indicator viewable within the cross-sectional image; and
determining, based on the cross-sectional image of the dial indicator, the position of the dial based on the position of the dial indicator viewable within the cross-sectional image.

16. The method of claim 15, wherein the valve device includes a housing having a reference indicator viewable through cross-sectional imaging of the housing, wherein the cross-sectional image of the valve device includes a position of the reference indicator viewable within the cross-sectional image, and wherein the method further comprises:
determining, based on the position of the reference indicator, an orientation of the valve device.

17. The method of claim 15, wherein the valve device includes a flow indicator positionable within a lumen distal to the dial, the flow indicator being moveable by flow of material through the lumen and viewable through cross-sectional imaging of the flow indicator, wherein the cross-sectional image of the valve device includes a position of the flow indicator viewable within the cross-sectional image, and wherein the method further comprises:
positioning the flow indicator within a lumen distal to the dial, the flow indicator being moveable by flow of material through the lumen; and
determining, based on a position of the flow indicator viewable through cross-sectional imaging, if material is flowing through the lumen.

18. The method of claim 17, wherein the flow indicator includes a magnetic-responsive element, the method further comprising:
returning the flow indicator to a non-occluding position by activation of the magnetic-responsive element of the flow indicator by an external magnetic device such that the flow indicator is drawn away from the distal lumen.

19. The method of claim 15, wherein the valve device includes a locking mechanism, that, when engaged, prevents alteration of the position of the dial, wherein the dial includes a magnetic-responsive element, and wherein the method further comprises:
disengaging the locking mechanism by activating the magnetic-responsive element to partially decouple the dial from a housing.

20. The method of claim 15, wherein the dial includes a magnetic-responsive element, and wherein the method further comprises:
activating the magnetic-responsive element of the dial by an external magnetic device; and
altering the position of the dial by rotation of the dial through the external magnetic device and the magnetic-responsive element.

21. The method of claim 15, further comprising:
positioning the valve device within a body in communication with a medical tube for controlling a flow of material through the medical tube.
